SEQUENCE LISTING (7 sequences)

SEQ ID NO: 1 - DNA, 22 bp, Mus musculus
  misc_feature 1..22: note = CrT Forward primer
  source 1..22: mol_type = genomic DNA
aggtttcctcaggttatagaga

SEQ ID NO: 2 - DNA, 21 bp, Mus musculus
  misc_feature 1..21: note = CrT Reverse primer
  source 1..21: mol_type = genomic DNA
ccctaggtgtatctaacatct

SEQ ID NO: 3 - DNA, 20 bp, Mus musculus
  misc_feature 1..20: note = CrT Reverse primer 1
  source 1..20: mol_type = genomic DNA
tcgtggtatcgttatgcgcc

SEQ ID NO: 4 - DNA, 27 bp, synthetic construct
  misc_feature 1..27: note = upstream 5 adaptor sequence 5 NTsc
  source 1..27: mol_type = other DNA
gtagcaacagctacaggcgcgcactcc

SEQ ID NO: 5 - DNA, 32 bp, synthetic construct
  misc_feature 1..32: note = downstream 3 adaptor sequence CTsc
  source 1..32: mol_type = other DNA
taatgagggatcccccgacctcgacctctggc

SEQ ID NO: 6 - protein, 6 aa, synthetic construct
  REGION 1..6: note = Histidine peptide tag
HHHHHH

SEQ ID NO: 7 - protein, 10 aa, synthetic construct
  REGION 1..10: note = Myc peptide tag
EQKLISEEDL

(19) United States
(12) Patent Application Publication
(10) Pub. No.: US 2025/0314665 A1
(43) Pub. Date: Oct. 9, 2025

(54) BIOMARKERS FOR CEREBRAL METABOLIC DISORDERS, AND DIAGNOSTIC METHODS USING THEREOF

(71) Applicants: COMMISSARIAT A L'ENERGIE ATOMIQUE ET AUX ENERGIES ALTERNATIVES, Paris (FR); University of Sharjah, Sharjah (AE)

(72) Inventors: Aloise MABONDZO, GIF SUR YVETTE (FR); Lea BROCA-BRISSON, GIF SUR YVETTE (FR); Narciso COSTA, GIF SUR YVETTE (FR); Anne-Cécile GUYOT, GIF SUR YVETTE (FR); Jean ARMENGAUD, Bagnols-Sur-Ceze-Cedex (FR); Rifat HAMOUDI, Sharjah (AE); Rania HARATI, Sharjah (AE)

(21) Appl. No.: 18/873,061

(22) PCT Filed: Jun. 12, 2023
(86) PCT No.: PCT/EP2023/065678

§ 371 Date: Dec. 9, 2024

(30) Foreign Application Priority Data
Jun. 13, 2022 (FR) 22305856

(51) Int. Cl. G01N 33/68

(52) U.S. Cl. CPC G01N 33/6896; C12Y 301/04011; G01N 2333/475; G01N 2333/916; G01N 2500/04; G01N 2800/28; G01N 2800/385; G01N 2800/52

(57) ABSTRACT

A method for diagnosing a cerebral creatine deficiency syndrome, the method comprising the steps of a) measuring an amount of at least one protein selected from BDNF, KIF1A, MeCP2, PLCB1, and a combination thereof, in an isolated biological sample, and b) comparing the measured amount obtained at step a) with a predetermined reference amount of said protein, where a difference between the measured amount and the predetermined reference amount is indicative of a cerebral creatine deficiency syndrome in said individual. At least one protein selected from BDNF, KIF1A, MeCP2, PLCB1, and a combination thereof, as a biomarker for use in a method for diagnosing a cerebral creatine deficiency syndrome.

### TECHNICAL FIELD

The present disclosure relates to biomarkers for cerebral metabolic disorders, and their use for diagnosis methods, for assessing the efficacy of therapeutic agents for the treatment of cerebral metabolic disorders, and for screening therapeutic agents for the prevention and/or the treatment of cerebral metabolic disorders. Cerebral metabolic disorders which may be concerned are congenital creatine deficiencies.

### TECHNICAL BACKGROUND

Intellectual disabilities and neurodevelopmental disorders represent significant health problem due to the heterogeneity of underlying causes and a lack of treatment options.

Creatine (Cr) transporter deficiency (CTD) is an X-linked inherited metabolic disease caused by SLC6A8 (Cr transporter; CrT; Braissant O, Henry H, Beard E, Uldry J. Creatine deficiency syndromes and the importance of creatine synthesis in the brain. Amino Acids 40, 1315-1324 (2011). Mutations of the gene, which moves Cr across the blood brain barrier and into neurons, preventing the transport of Cr into the brain. Cr is essential for proper brain function, has a crucial role in energy storage and transmission, and has anti-apoptotic, antioxidant, neuroprotector and neuromodulator effects (van de Kamp, J. M., Mancini, G. M. & Salomons, G. S. X-linked creatine transporter deficiency: clinical aspects and pathophysiology. Journal of Inherited Metabolic Disease 37, 715-733, (2014)). Patients suffering from CTD have autistic-spectrum disorder with moderate to severe intellectual disabilities, behavioral disorders, development delay and seizures (Stockler, S., Schutz, P. W. & Salomons, G. S. Cerebral creatine deficiency syndromes: clinical aspects, treatment and pathophysiology. Subcell Biochem 46, 149-166, (2007); Salomons, G. S. et al. X-linked creatine-transporter gene (SLC6A8) defect: A new creatine-deficiency syndrome. American Journal of Human Genetics 68, 1497-1500, (2001); and DesRoches, C. L. et al. Estimated carrier frequency of creatine transporter deficiency in females in the general population using functional characterization of novel missense variants in the SLC6A8 gene. Gene 565, 187-191, (2015)). Slc6a8^{-/y} (CrT KO) mice show a marked Cr depletion in the brain and have significant cognitive impairment and autistic-like behavior, recapitulating the key clinical features of human CTD.

Several combinations of nutritional supplements have been attempted with very limited success as therapeutic approaches for CTD (Bruun, T. U. J. et al. Treatment outcome of creatine transporter deficiency: international retrospective cohort study. Metabolic Brain Disease 33, 875-884, (2018); Valayannopoulos, V. et al. Functional and electrophysiological characterization of four non-truncating mutations responsible for creatine transporter (SLC6A8) deficiency syndrome. Journal of Inherited Metabolic Disease 36, 103-112, (2013); and Jaggumantri, S. et al. Treatment of Creatine Transporter (SLC6A8) Deficiency With Oral S-Adenosyl Methionine as Adjunct to L-arginine, Glycine, and Creatine Supplements. Pediatr Neurol 53, 360-363.e362, (2015)). Dodecyl creatine ester (DCE) has proven to be a promising a therapeutic option based on preclinical in vitro and in vivo data (Trotier-Faurion, A. et al. Synthesis and Biological Evaluation of New Creatine Fatty Esters Revealed Dodecyl Creatine Ester as a Promising Drug Candidate for the Treatment of the Creatine Transporter Deficiency. Journal of Medicinal Chemistry 56, 5173-5181, (2013); Trotier-Faurion, A. et al. Dodecyl creatine ester and lipid nanocapsule: a double strategy for the treatment of creatine transporter deficiency. Nanomedicine 10, 185-191, (2015); and Ullio-Gamboa, G. et al. Dodecyl creatine ester-loaded nanoemulsion as a promising therapy for creatine transporter deficiency. Nanomedicine 14, 1579-1593, doi:10.2217/nnm-2019-0059 (2019)). To provide the rational basis of this therapeutic solution to the treatment of CTD patients, the present study highlights several molecular players disrupted in the brain of Slc6a8^{-/y} mice (Raffaele M, et al. Novel translational phenotypes and biomarkers for creatine transporter deficiency. Brain Commun, (2020)) and are modulated by DCE treatment. Further, it is shown that these protein changes correlate to cognitive performance in mouse model of CTD.

Cerebral creatine deficiency in brain MR spectroscopy (1H-MRS) is the characteristic hallmark of all CCDS. Diagnosis of CTD relies on measurement of creatinine in brain by MR spectroscopy and on molecular genetic testing of the gene involved, SLC6A8. If molecular genetic test results are inconclusive, creatine uptake in cultured fibroblasts can be assessed. MR spectroscopy used to track brain creatine spike cannot be used to monitor the regulation of brain proteins involved in the pathophysiology of cerebral metabolic diseases. Further, MR spectroscopy is quite cumbersome and cannot easily and readily implemented in routine practice to follow the evolution of the disorder or the efficacy of a therapeutic agent.

Therefore, there is a need for biomarkers of cerebral metabolic diseases, such as cerebral creatine deficiency syndrome, suitable for systemic measures.

There is a need for biomarkers able to connect cognitive functions and physiopathology of cerebral metabolic diseases, such as cerebral creatine deficiency syndrome.

There is a need for biomarkers which can be easily dosed in biological samples.

There is a need for biomarkers which can allow discriminating cerebral creatine deficiency syndrome, such as creatine transporter (CRTR) deficiency, from other cerebral metabolic disorders.

There is a need for biomarkers which can allow the monitoring of a treatment in an individual suffering from a cerebral creatine deficiency syndrome, such as creatine transporter (CRTR) deficiency.

There is a need for biomarkers which can be used as diagnostic tools as well as tools for monitoring the efficacy of a treatment of a cerebral creatinine syndrome, or monitoring the evolution of a cerebral creatinine syndrome, or else for screening drug candidates.

The present disclosure has for purpose to satisfy all or part of those needs.

### SUMMARY

In one of its objects, the present disclosure relates to a method for diagnosing a cerebral creatine deficiency syndrome in an individual in need thereof, said method comprising at least the steps of:
a) measuring an amount of at least one protein selected from BDNF, KIF1A, MeCP2, PLCB1, and a combination thereof, in an isolated biological sample obtained from said individual,
b) comparing the amount measured at step a) with a predetermined reference value,
wherein a difference between the measured amount and the predetermined reference value is indicative of a cerebral creatine deficiency syndrome in said individual.

In some embodiments, step a) comprises measuring an amount of the protein BDNF.

In some embodiments, step a) comprises measuring an amount of each protein KIF1A, MeCP2 and PLCB1. In some embodiments, step a) comprises further measuring an amount of the protein BDNF.

In another of its objects, the present disclosure relates to a method for diagnosing a cerebral creatine deficiency syndrome in an individual in need thereof, said method comprising the steps of:
a) measuring an amount of each protein KIF1A, MeCP2, PLCB1 in an isolated biological sample obtained from said individual,
b) comparing each amount measured at step a) with a predetermined reference value,
wherein a difference between the measured amounts and the predetermined reference values may be indicative of a cerebral creatine deficiency syndrome in said individual.

At step b), each measure amount is compared with a corresponding predetermined reference value.

Surprisingly, as shown in the Examples section, the inventors have observed that it was possible to correlate the effectiveness of creatine dodecyl ester with behavioral changes in a mouse model of creatine transporter deficiency, Slc6a8^{-/y} mice, and with the modulation of the expression of some key proteins involved in autism, cerebellar ataxia, Rett syndrome, axonal neuropathy, or leukodystrophy. The impacted proteins are usable as biomarkers of cerebral metabolic disorders, such as a cerebral creatine deficiency syndrome, and as biomarkers of the effectiveness of the treatment of those disorders. In some embodiments, the identified biomarkers may be used in diagnosis methods for CTD, for selecting new therapeutic agents and for monitoring the efficacy of therapeutic agents for preventing and/or treating cerebral creatine deficiency syndromes.

The results presented in the Examples section show that dodecyl creatine ester delivery in creatine transporter deficient mice restores both behavioral traits and protein levels in the different brain regions.

The inventors have surprisingly observed that the amounts of KIF1A tended to be upregulated in the tested brain regions (cortex, hippocampus, cerebellum, and brain stem) of a mouse model of a cerebral creatine deficiency syndrome i.e., a creatine transporter (CRTR) deficiency, compared with healthy individuals, while the amounts of MeCP2 and PLCB1 tended to be downregulated. Furthermore, the inventors have observed that a treatment with DCE in the mouse model was able to restore amounts of proteins comparable to the wildtype animals.

The inventors have also observed that in a CTD mouse model, DCE-rescued mice resulted in higher pro-BDNF/BDNF level. A high pro-BDNF/BDNF level which is linked to cognitive function improvement. This observation pointed to the use of BDNF as biomarker for cerebral creatine deficiency syndromes.

Further, the inventors have surprisingly observed that the amounts of IGSF8 tended to be downregulated in the tested brain regions (cortex, hippocampus, cerebellum, and brain stem) of a mouse model of a cerebral creatine deficiency syndrome i.e., a creatine transporter (CRTR) deficiency, compared with healthy individuals, while the amounts of LMNB1 and FABP7 tended to be upregulated. Furthermore, the inventors have observed that a treatment with DCE in the mouse model was able to restore amounts of proteins comparable to the wildtype animals.

The inventors have surprisingly observed that the amounts of NCAM1, DCLK1, L1CAM, PURB and MYO5 tended to be upregulated in the tested brain regions (cortex, hippocampus, cerebellum, and brain stem) of a mouse model of a cerebral creatine deficiency syndrome i.e., a creatine transporter (CRTR) deficiency, compared with healthy individuals, while the amounts of PI4K-A, ANK1 and ANXA5 tended to be downregulated. Also, the inventors have observed that, DCE treatment significantly normalized their levels in the corresponding brain regions.

In some embodiments, the cerebral metabolic disorders considered herein may be cerebral creatine deficiency syndromes, leukodystrophy, cerebellar ataxia, intellectual deficits, bipolar syndrome, autistic syndromes, or astrocytopathies.

According to another of its objects, the present disclosure relates to a method for monitoring a therapeutic efficacy of a therapeutic treatment proposed for preventing and/or treating a cerebral creatine deficiency syndrome in an individual in need thereof, said method comprising the steps of:
a) measuring an amount of at least one each protein selected from BDNF, KIF1A, MeCP2, and PLCB1, and a combination thereof, in an isolated biological sample obtained from said individual before administration of said therapeutic treatment,
b) measuring an amount of at least one each protein selected from BDNF, KIF1A, MeCP2, and PLCB1, and a combination thereof, in an isolated biological sample obtained from said individual after administration of said therapeutic treatment, the protein or combination of proteins of step a) and b) being the same,
c) comparing the amounts measured at step a) with the amounts measured at step b),
wherein a difference between the measured amounts at step a) and at step b) is indicative of a therapeutic efficacy of said therapeutic treatment on said cerebral creatine deficiency syndrome.

In some embodiments, steps a) and b) comprise measuring an amount of the protein BDNF.

In some embodiments, steps a) and b) comprise measuring an amount of each protein KIF1A, MeCP2 and PLCB1. In some embodiments, steps a) and b) comprise further measuring an amount of the protein BDNF.

According to another of its objects, the present disclosure relates to a method for monitoring a therapeutic efficacy of a therapeutic treatment proposed for preventing and/or treating a cerebral creatine deficiency syndrome in an individual in need thereof, said method comprising the steps of:
a) measuring an amount of each protein KIF1A, MeCP2, and PLCB1 in an isolated biological sample obtained from said individual before administration of said therapeutic treatment,
b) measuring an amount of each protein KIF1A, MeCP2, and PLCB1 in an isolated biological sample obtained from said individual after administration of said therapeutic treatment,
c) comparing the amounts measured at step a) with the amounts measured at step b),
wherein a difference between the measured amounts obtained at step a) and at step b) is indicative of a therapeutic efficacy of said therapeutic treatment on said cerebral creatine deficiency syndrome.

According to another of its objects, the present disclosure relates to a method for selecting a candidate therapeutic agent for preventing and/or treating a cerebral creatine deficiency syndrome in an individual in need thereof, said method comprising the steps of:
a) measuring an amount of at least one protein selected from BDNF, KIF1A, MeCP2, PLCB1, and a combination thereof, in an isolated biological sample obtained from a biological model of a cerebral creatine deficiency syndrome before contacting said biological model with said candidate therapeutic agent,
b) measuring an amount of at least one protein selected from BDNF, KIF1A, MeCP2, PLCB1, and a combination thereof, in an isolated biological sample obtained from said biological model of step a) after contacting said biological model with said candidate therapeutic agent, the protein or combination of proteins of step a) and b) being the same,
c) comparing the amounts measured at step a) with the amounts measured at step b), and
d) selecting a candidate therapeutic agent for which a difference between the measured amounts obtained at step a) and at step b) is indicative of a therapeutic efficacy of said candidate therapeutic agent on said cerebral creatine deficiency syndrome.

In some embodiments, steps a) and b) comprise measuring an amount of the protein BDNF.

In some embodiments, steps a) and b) comprise measuring an amount of each protein KIF1A, MeCP2 and PLCB1. In some embodiments, steps a) and b) comprise further measuring an amount of the protein BDNF.

According to another of its objects, the present disclosure relates to a method for selecting a candidate therapeutic agent for preventing and/or treating a cerebral creatine deficiency syndrome in an individual in need thereof, said method comprising the steps of:
a) measuring an amount of each protein KIF1A, MeCP2, and PLCB1 in an isolated biological sample obtained from a biological model of a cerebral creatine deficiency syndrome before contacting said biological model with said candidate therapeutic agent,
b) measuring an amount of each protein KIF1A, MeCP2, and PLCB1 in an isolated biological sample obtained from said biological model of step a) after contacting said biological model with said candidate therapeutic agent,
c) comparing the amounts measured at step a) with the amounts measured at step b), and
d) selecting a candidate therapeutic agent for which a difference between the measured amounts obtained at step a) and at step b) is indicative of a therapeutic efficacy of said candidate therapeutic agent on said cerebral creatine deficiency syndrome.

In one of its objects, the present disclosure relates to a method for monitoring an evolution of a cerebral creatine deficiency syndrome in an individual in need thereof, said method comprising the steps of:
a) measuring an amount of at least one protein selected from BDNF, KIF1A, MeCP2, PLCB1, and a combination thereof, in an isolated biological sample obtained from said individual at a first time,
b) measuring an amount of at least one protein selected from BDNF, KIF1A, MeCP2, PLCB1, and a combination thereof, in an isolated biological sample obtained from said individual at a second time, subsequent to the first time, the protein or combination of proteins of step a) and b) being the same,
c) comparing the measured amounts obtained at step a) and at step b),
wherein a difference between the measured amounts may be indicative of an improvement or an aggravation of the cerebral creatine deficiency syndrome in said individual.

In some embodiments, steps a) and b) comprise measuring an amount of the protein BDNF.

In some embodiments, steps a) and b) comprise measuring an amount of each protein KIF1A, MeCP2 and PLCB1. In some embodiments, steps a) and b) comprise further measuring an amount of the protein BDNF.

According to another of its objects, the present disclosure relates to a method for monitoring an evolution of a cerebral creatine deficiency syndrome in an individual in need thereof, said method comprising the steps of:
a) measuring an amount of each protein KIF1A, MeCP2, and PLCB1 in an isolated biological sample obtained from said individual at a first time,
b) measuring an amount of each protein KIF1A, MeCP2, and PLCB1 in an isolated biological sample obtained from said individual at a second time, subsequent to the first time,
c) comparing the amounts measured at step a) with the amounts measured at step b),
wherein a difference between the measured amounts may be indicative of an improvement or an aggravation of the cerebral creatine deficiency syndrome in said individual.

In some embodiments, the methods of the disclosure may further comprise a measure an amount of at least one protein from the group FABP7, LMNB1, and IGSF8, and combinations thereof.

The methods may further comprise a measure of an amount of at least one protein selected among NCAM1, ANXA5, DCLK1, L1CAM, PI4K-A, MYO5, ANK1 and PURB, and combinations thereof.

In some embodiments, the cerebral creatine deficiency syndrome may be a creatine transporter (CRTR) deficiency.

The biological sample may be selected from the group consisting of blood, plasma, serum, cerebrospinal fluid, or is a brain organoid prepared by dedifferentiation and reprogramming of fibroblast cells obtained from said individual.

In some embodiments, a biological sample may be selected from the group consisting of blood, plasma, and serum sample.

According to another of its objects, the present disclosure relates to a use of at least one protein selected from BDNF, KIF1A, MeCP2, PLCB1, and a combination thereof, as a biomarker a cerebral creatine deficiency syndrome.

According to another of its objects, the present disclosure relates to a use of at least the protein BDNF as a biomarker a cerebral creatine deficiency syndrome.

According to another of its objects, the present disclosure relates to a use of a set of proteins comprising KIF1A, MeCP2, and PLCB1 as biomarker a cerebral creatine deficiency syndrome. In an embodiment, the biomarker further may comprise the protein BDNF.

According to another of its objects, the present disclosure relates to a biomarker for use in a method for diagnosing a cerebral creatine deficiency syndrome, wherein the biomarker comprises at least one protein selected from BDNF, KIF1A, MeCP2, PLCB1, and a combination thereof.

According to another of its objects, the present disclosure relates to a biomarker for use in a method for diagnosing a cerebral creatine deficiency syndrome, wherein the biomarker comprises at least the protein BDNF.

According to another of its objects, the present disclosure relates to a biomarker for use in a method for diagnosing a cerebral creatine deficiency syndrome, wherein the biomarker is of a set of proteins comprising KIF1A, MeCP2, and PLCB1. In an embodiment, the biomarker further may comprise the protein BDNF.

According to another of its objects, the present disclosure relates to at least one protein selected from BDNF, KIF1A, MeCP2, PLCB1, and a combination thereof, as a biomarker for use in a method for diagnosing a cerebral creatine deficiency syndrome.

According to another of its objects, the present disclosure relates to at least the protein BDNF as a biomarker for use in a method for diagnosing a cerebral creatine deficiency syndrome.

According to another of its objects, the present disclosure relates to KIF1A, MeCP2, and PLCB1 as a biomarker for use in a method for diagnosing a cerebral creatine deficiency syndrome. In an embodiment, the biomarker further may comprise the protein BDNF.

In some embodiments, the uses or the biomarker of the disclosure may further comprise at least one protein from the group FABP7, LMNB1, and IGSF8, and combinations thereof, and/or of at least one protein from the group NCAM1, ANXA5, DCLK1, L1CAM, PI4K-A, MYO5, ANK1 and PURB, and combinations thereof.

In the uses of the disclosure, the cerebral creatine deficiency syndrome may be a creatine transporter (CRTR) deficiency.

According to another of its objects, the present disclosure relates to a kit for diagnosing a cerebral creatine deficiency syndrome, said kit comprising means for measuring an amount of at least one protein selected from BDNF, KIF1A, MeCP2, PLCB1, and a combination thereof, in an isolated biological sample.

In an embodiment, the kit comprises means for measuring an amount of the protein BDNF.

In an embodiment, the kit comprises means for measuring an amount of each protein KIF1A, MeCP2, and PLCB1. In an embodiment, the kit further comprises means for measuring an amount of the protein BDNF.

According to another of its objects, the present disclosure relates to a kit for diagnosing a cerebral creatine deficiency syndrome, said kit comprising means for measuring an amount of each protein KIF1A, MeCP2, and PLCB1 in an isolated biological sample.

In some embodiments, the means for determining the amount of said proteins are configured for performing an immunoassay and/or a mass-spectrometric-based assay.

In some embodiments, a kit may comprise an instruction to compare the measured amounts of the proteins with predetermined reference values.

According to another of its objects, the present disclosure relates to a use of a kit according to the disclosure for diagnosing a cerebral creatine deficiency syndrome.

In some embodiments, uses and methods disclosed herein are in vitro uses and methods.

According to another of its objects, the present disclosure relates to a method of diagnosing and treating an individual susceptible to suffer from a cerebral creatine deficiency syndrome, the method comprising the steps of:
a) measuring an amount of at least one protein selected from BDNF, KIF1A, MeCP2, PLCB1, and a combination thereof, in an isolated biological sample obtained from said individual,
b) comparing the measured amount obtained at step a) with a predetermined reference amount of said protein,
wherein a difference between the measured amount and the predetermined reference amount is indicative of a cerebral creatine deficiency syndrome in said individual, and
c) administering to said individual to whom a difference between the measured amounts and the predetermined reference amounts is observed a therapeutic agent proposed preventing and/or treating a cerebral creatine deficiency syndrome.

In some embodiments, step a) comprises measuring an amount of the protein BDNF.

In some embodiments, step a) comprises measuring an amount of each protein KIF1A, MeCP2 and PLCB1. In some embodiments, step a) comprises further measuring an amount of the protein BDNF.

According to another of its objects, the present disclosure relates to a method of diagnosing and treating an individual susceptible to suffer from a cerebral creatine deficiency syndrome, the method comprising the steps of:
a) measuring an amount of each protein KIF1A, MeCP2, and PLCB1 in an isolated biological sample obtained from said individual,
b) comparing the measured amounts obtained at step a) with a set of predetermined reference amounts of said proteins,
wherein a difference between the measured amounts and the predetermined reference amounts is indicative of a cerebral creatine deficiency syndrome in said individual, and
c) administering to said individual to whom a difference between the measured amounts and the predetermined reference amounts is observed a therapeutic agent proposed preventing and/or treating a cerebral creatine deficiency syndrome.

In some embodiments, step a) comprises further measuring an amount of the protein BDNF.

### BRIEF DESCRIPTION OF THE FIGURES

FIG. 1: represents a flowchart diagram depicting the workflow used to identify the key driver proteins linked with cognitive functions and regulated by dodecyl creatine ester treatment in creatine transporter deficiency pathogenesis. The raw proteomics data was normalized using the Variance Stabilizing Normalization function and an unsupervised filter was applied. The differentially abundant proteins between the different regions were identified using Reproducibility Optimized Statistical Testing. The assessment of data was carried out using variance bioinformatic tools. Venn diagram analysis was performed, and the proteins significantly altered by the CrT deficiency compared to the wild-type, and by the treatment compared to vehicle were selected for a subsequent pathway analysis. Pathway analysis was performed using Enrichr with a cut-off value of p lower than 0.05 and the proteins identified by the different pathways and diseases were selected. The normalized values of the selected proteins were subjected to the statistical analysis comparing the groups and the correlation with the cognitive tests. Correlation analysis was performed using a stepwise regression model between the 14 selected proteins affected by both the CrT deficiency and the DCE treatment with the cognitive tests. The quality of the separation of the data between the various groups being compared was assessed using the reproducibility plots and the principal component analysis (PCA). The differentially expressed proteins were visualized using volcano plots. The degree of separation between the groups was assessed using unsupervised hierarchical clustering.

FIG. 2: represent the cognitive studies and comparison of proteomic signatures between the different experimental groups across the different brain regions. FIGS. 2A & 2B: represent the impairment of object recognition in CrT KO mice measures in object recognition test (ORT). FIG. 2A represents the object discrimination index and FIG. 2B represents the percent time with the novel object showing the preference for the novel object in WT (wild-type) and DCE-treated, but not vehicle-treated KO, mice. FIG. 2C represents the early deficiency of working and spatial memory in CrT KO mice measured in the Y-maze. CrT KO mice showed an alteration of the spontaneous alternation performance in the Y-maze test which is significantly improved by DCE treatment. Data are the mean±s.e.m. Statistical analysis performed by one-way ANOVA followed by Tukey post hoc test. *p<0.05; **p<0.001; ns=non-significant.

FIG. 3: represents the fourteen proteins significantly altered by the mutation and the treatment with the pathways and diseases identified by Enrichr analysis. Fourteen proteins were found to be significantly altered by the mutation and the treatment. Pathway analysis was performed on these 14 proteins using Enrichr pathway analysis using gene set enrichment carried out using Enrichr focusing on the following sets: BioCarta_2016, Elsevier_Pathway_Collection, GO_Biological_Process_2018, GO_Molecular_Function_2018, KEGG_2019_Human, KEGG_2019_Mouse, MSigDB_Hallmark_2020, WikiPathways_2019_Mouse, WikiPathways_2019_Human, ClinVar_2019, DisGeNET, Jensen_DISEASES, OMIM_Disease. Relevant pathways were selected based on a p<0.05 cut-off.

FIGS. 4A and B: Tables presenting proteins showing a significant correlation with cognitive performance in discrimination index (FIG. 4A) and Y-maze test (FIG. 4B).

FIG. 5A-J: PLCB1 signaling and data summary. FIG. 5A-C: 0 days after DCE treatment of CrT KO mice, PLCB1 protein abundance evidenced by Western blot was significantly increased in the brain cortex (FIG. 5A), hippocampus (FIG. 5B) and cerebellum (FIG. 5G). FIG. 5D-E: Western blot results of IKbα (FIG. 5D) and IKbβ (FIG. 5E) showing that DCE promotes IKbα transcriptional factor expression but not IKβi. Data are the mean±s.e.m. (n=8/groups). FIG. 5F-H: Western blot results showing that KIF1A was significantly increased in the cortex (FIG. 5F), hippocampus (FIG. 5G) and cerebellum (FIG. 5H) of vehicle CrT KO mice compared to the WT mice, while DCE treatment rescued this overexpression in the three brain regions. FIG. 5I-J: Western blot results showing that Pro-BDNF/BDF ratio and PSD95 are significantly regulated in the cortex of vehicle CrT KO mice. Statistical analysis was performed by one-way ANOVA followed by the Tukey's post hoc test. *p≤0.05; **p≤0.001; ***p≤0.0001; ns=non-significant.

### BRIEF DESCRIPTION OF THE SEQUENCES

SEQ ID NO: 1 refers to AGGTTTCCTCAGGTTATAGAGA forward primer for SLC6A8 gene.

SEQ ID NO: 2 refers to CCCTAGGT GTATCTAACATCT reverse primer for SLC6A8 gene.

SEQ ID NO: 3 refers to TCGTGGTATCGTTATGCGCC reverse primer 1 for SLC6A8 gene.

SEQ ID NO: 4 refers to the upstream 5′ adaptor sequence 5′ NTsc GTAGCAACAGCTACAGGCGCGCACTCC.

SEQ ID NO: 5 refers to the downstream 3′ adaptor sequence CTsc TAATGAGGGATCCCCCGACCTCGACCTCTGGC.

SEQ ID NO: 6 refers to a C-terminal Histidine (HHHHHH) peptide tag.

SEQ ID NO: 7 refers to a C-terminal Myc (EQKLISEEDL) peptide tag.

### DETAILED DESCRIPTION

### Definitions

Unless otherwise defined herein, scientific and technical terms used in connection with the present disclosure shall have the meanings that are commonly understood by those of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular.

Units, prefixes, and symbols are denoted in their Système International des Unités (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is understood that wherever aspects are described herein with the language “comprising,” otherwise analogous aspects described in terms of “comprising only”, “consisting of” and/or “consisting essentially of” are also provided.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Within the disclosure, the terms “amount” or “measured amount” intends to refer to an absolute or relative amount or concentration of a compound, e.g. a protein, the presence or absence of a compound, a range of amounts or concentrations of a compound, a minimum and/or maximum amount or concentration of a compound, a mean amount or concentration of a compound, and/or a median amount or concentration of a compound; and, in addition, when analyzing a combination of compounds also the ratios of absolute or relative amounts or concentrations of two or more compounds with respect to each other may be measured. In some embodiments, measured amounts may be transformed by variance-stabilizing transformation, according to known statistical methods, before being compared to predetermined reference values.

Within the disclosure, the expression “sample” or “biological sample” refers to biological material isolated from the subject. Examples for biological samples are any suitable biological tissue or fluid such as of blood, plasma, serum, or cerebrospinal fluid. Also, a biological sample may be a brain organoid prepared by dedifferentiation and reprogramming of fibroblast cells obtained from said individual.

Within the disclosure, the term “biomarker” intends to mean a compound, such as a protein, or a set of compounds, taking part in a particular biological process in an individual and which can be measured in the body, its products, or isolated biological sample, and influence or predict the incidence of outcome or disease. The biomarker might be an intermediate or a product of a biological process. A biomarker may be a compound, a measured amount of the compound or a result of comparison between a measured amount of the compound and a predetermined value of reference.

Within the disclosure, the expression “cerebral metabolic disorder” intends to refer to metabolic disorder which disrupts a normal metabolic process. In other words, a cerebral metabolic disorder is a disturbance of the internal homeostasis of the brain, brought about by an abnormal change in the rate of one or more critical metabolic processes. Therefore, a metabolic disorder may be characterized by abnormal chemical reactions in an individual's brain, which alter the normal metabolic process. It can be the result of an inherited gene abnormality. Because of the disruption of a normal metabolic process an overproduction and/or underproduction of metabolic products, like specific biomarkers, in the individual may be the consequence. The metabolic imbalance may result in a metabolic disorder. The definition of metabolic disorder should not be interpreted too broadly. Even infectious diseases, such as those caused by viruses and bacteria exert their clinical effects by altering the internal homeostasis of the body, but these diseases are not primarily metabolic in character.

Within the disclosure, the expression “cerebral creatine deficiency syndrome” intends to refer to inborn errors of creatine metabolism. This syndrome includes the two creatine biosynthesis disorders, guanidinoacetate methyltransferase (GAMT) deficiency and L-arginine: glycine amidinotransferase (AGAT) deficiency, and the creatine transporter (CRTR) deficiency. Intellectual disability and seizures are common to all three CCDS. Onset of the syndrome is between ages three months and three years.

Within the disclosure, the expression “creatine (Cr) transporter deficiency (CTD)” intends to refer to a set of disorders resulting from mutations in the coding sequence of the creatine transporter CRT1. CTD is characterized by the absence of cerebral creatine. The phenotype of CTD deficiency ranges from mild intellectual disability and speech delay to severe intellectual disability, seizures, movement disorder, and behavior disorder.

Within the disclosure, the term “individual” is used interchangeably with patient and intends to refer to any human or non-human animal. The term non-human animal includes all vertebrates, e.g., mammals and non-mammals, such as non-human primates, monkeys, rats, mice, sheep, dogs, cows, cats, horses, rabbits, pigs, chicken, amphibians, reptiles, rodents, etc. “Healthy reference individual” means a subject which is on a normal, healthy state and from which a value of reference for a given biomarker may be obtained and use as “predetermined reference value”.

Within the disclosure, the term “isolated” used with respect to a biological sample, a protein, or a gene intends to refer to an element that is not in its natural milieu. No particular level of purification is required. For example, an isolated biological sample or protein can simply be removed from its native or natural environment.

Within the disclosure, the expression “predetermined reference value” or “reference value” intends to refer to a threshold value or amount, a reference range or a cut-off value or amount of a protein or a biomarker or an index defined by set of proteins or biomarkers that can be determined experimentally, empirically, or theoretically and by comparing with which, a diagnosis of a cerebral creatine deficiency syndrome can be made. A threshold value or amount can also be arbitrarily selected based upon the existing experimental and/or clinical conditions, as would be recognized by a person of ordinary skilled in the art. A predetermined reference value or amount may be selected to maximize sensitivity while keeping the specificity above a user-defined threshold. A predetermined reference value or amount can also be selected to maximize specificity while keeping the sensitivity above a user-defined threshold, for example, 80% sensitivity. A predetermined reference value or amount can be a threshold amount or value of a protein or a biomarker or a threshold of an index defined by a set of proteins biomarkers obtained from a population of healthy individuals.

Within the disclosure, “preventing”, “prevent” or “prevention” include reduction of risk and/or severity of a condition or disorder. The terms “treatment,” “treat,” “treating,” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of individuals at risk of contracting or suffering from a disease or suspected to have contracted or to suffer from a disease, as well as individuals who are ill or have been diagnosed as suffering from a disease or medical condition. The term does not necessarily imply that an individual is treated until total recovery. The terms “treatment,” “treat,” and “treating” also refer to the maintenance and/or promotion of health in an individual not suffering from a disease but who may be susceptible to the development of an unhealthy condition. The terms “treatment,” “treat,” “treating,” and “to alleviate” are also intended to include the potentiation or otherwise enhancement of one or more primary prophylactic or therapeutic measure.

A “therapeutic regimen,” “therapy” or “treatment(s)” intends to refer to all clinical management of an individual and interventions, whether biological, chemical, physical, behavioral or a combination thereof, intended to sustain, ameliorate, improve, or otherwise alter the condition of a cerebral creatine deficiency syndrome in an individual. These terms may be used synonymously herein. Treatments include but are not limited to occupational, speech, physical and behavioral therapies as well as administration of prophylactics or therapeutic agents known as efficacious or proposed to be efficacious in preventing, delaying the onset of, ameliorating or curing a cerebral creatine deficiency syndrome. A “response to treatment” includes an individual's response to any of the above-described treatments, whether biological, chemical, physical, behavioral or a combination of the foregoing. A “course of treatment” relates to the dosage, duration, extent, etc. of a particular treatment or therapeutic regimen.

As used herein, the expression “statistically different” or “significantly different” refers to that an observed alteration is greater than what would be expected to occur by chance alone (e.g., a “false positive”). Statistical significance can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than or equal to 0.05.

Within the disclosure, the term “substantially” used in conjunction with a feature, e.g., “an amount equal to or substantially equal to”, of the disclosure intends to define a set of embodiments related to this feature which are largely but not wholly similar to this feature.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### Biomarkers

### Proteins as Biomarkers

In some embodiments, the disclosure relates to the protein BDNF used as a biomarker. It can be used as biomarker of a cerebral creatine deficiency syndrome.

In some embodiments, the disclosure relates to at least one protein selected from BDNF, KIF1A, MeCP2, PLCB1, and combinations thereof, used as a biomarker. They can be used as biomarker of a cerebral creatine deficiency syndrome.

In some embodiments, the disclosure relates to the proteins KIF1A, MeCP2, and PLCB1 used as a biomarker. They can be used as biomarker of a cerebral creatine deficiency syndrome.

In some embodiments, a biomarker may further comprise at least one protein from the group FABP7, LMNB1, and IGSF8, and combinations thereof.

In some embodiments, a biomarker may further comprise at least one protein selected among NCAM1, ANXA5, DCLK1, L1CAM, PI4K-A, MYO5, ANK1 and PURB, and combinations thereof.

In some embodiments, a protein is a human protein.

A biomarker may be an amount of a protein. In some embodiments, a biomarker may be an observed difference between an amount of protein measured in an individual suspected to suffer from a cerebral creatine deficiency syndrome and a predetermined reference value of amount of said protein.

BDNF, or brain derived neurotrophic factor, also known as ANON2 or BULN2, is a member of the nerve growth factor family of proteins. Alternative splicing of the gene transcript results in multiple transcript variants, at least one of which encodes a preproprotein that is proteolytically processed to generate the mature protein. Binding of this protein to its cognate receptor promotes neuronal survival in the adult brain. Expression of this gene is reduced in Alzheimer's, Parkinson's, and Huntington's disease patients. This gene may play a role in the regulation of the stress response and in the biology of mood disorders.

KIF1A, or kinesin family member 1A, also known as ATSV; MRD9; HSN2C; SPG30; UNC104; C2orf20; NESCAVS, is a member of the kinesin family and functions as an anterograde motor protein that transports membranous organelles along axonal microtubules. Mutations at this locus have been associated with spastic paraplegia-30 and hereditary sensory neuropathy IIC.

MECP2, or methyl-CpG binding protein 2, also known as RS, RTS, RTT, PPMX, MRX16, MRX79, MRXSL, AUTSX3, MRXS13, is a nuclear protein comprising a methyl-CpG binding domain (MBD) and is capable of binding specifically to methylated DNA. MECP2 can repress transcription from methylated gene promoters. MECP2 is X-linked and subject to X inactivation. MECP2 is dispensible in stem cells but is essential for embryonic development. MECP2 gene mutations are the cause of most cases of Rett syndrome, a progressive neurologic developmental disorder and one of the most common causes of cognitive disability in females.

PLCB1, phospholipase C beta 1, also known as DEE12, PLC-I, EIEE12, PI-PLC, PLC154, PLCB1A, PLCB1B, PLC-154, PLC-beta-1, catalyzes the formation of inositol 1,4,5-trisphosphate and diacylglycerol from phosphatidylinositol 4,5-bisphosphate. This reaction uses calcium as a cofactor and plays an important role in the intracellular transduction of many extracellular signals.

FABP7, or fatty acid-binding protein (FABP) 7, also known as MRG, BLBP, FABPB, B-FABP, is a small cytosolic protein that enhance intracellular transfer of fatty acids and have a stimulatory effect on enzymes involved in the processes of fatty acid metabolism. This protein is important in the establishment of the radial glial fiber in the developing brain.

LMNB1, or lamin B1, also known as LMN, ADLD, LMN2, LMNB, MCPH26, is a component of the nuclear lamina. A duplication of this gene is associated with autosomal dominant adult-onset leukodystrophy (ADLD).

IGSF8, or immunoglobulin superfamily member 8, also known as EWI2, PGRL, CD316, EWI-2, KCT-4, CD81P3, LIR-D1, a member the EWI subfamily of the immunoglobulin protein superfamily. Members of this family contain a single transmembrane domain, an EWI (Glu-Trp-Ile)-motif and a variable number of immunoglobulin domains. This protein interacts with the tetraspanins CD81 and CD9 and may regulate their role in certain cellular functions including cell migration and viral infection. The encoded protein may also function as a tumor suppressor by inhibiting the proliferation of certain cancers.

NCAM1, neural cell adhesion molecule 1, also known as CD56, NCAM, MSK39, is a cell adhesion protein which is a member of the immunoglobulin superfamily. The protein is involved in cell-to-cell interactions as well as cell-matrix interactions during development and differentiation. The encoded protein plays a role in the development of the nervous system by regulating neurogenesis, neurite outgrowth, and cell migration. This protein is also involved in the expansion of T lymphocytes, B lymphocytes and natural killer (NK) cells which play an important role in immune surveillance. This protein plays a role in signal transduction by interacting with fibroblast growth factor receptors, N-cadherin and other components of the extracellular matrix and by triggering signalling cascades involving FYN-focal adhesion kinase (FAK), mitogen-activated protein kinase (MAPK), and phosphatidylinositol 3-kinase (PI3K).

ANXA5, or annexin A5, also known as PP4, ANX5, ENX2, RPRGL3, HEL-S-7, belongs to the annexin family of calcium-dependent phospholipid binding proteins some of which have been implicated in membrane-related events along exocytotic and endocytotic pathways. Annexin 5 is a phospholipase A2 and protein kinase C inhibitory protein with calcium channel activity and a potential role in cellular signal transduction, inflammation, growth and differentiation. Annexin 5 has also been described as placental anticoagulant protein I, vascular anticoagulant-alpha, endonexin II, lipocortin V, placental protein 4 and anchorin CII.

DCLK1, doublecortin like kinase 1, also known as CL1, DCLK, CLICK1, DCDC3A, DCAMKL1, is a member of the protein kinase superfamily and the doublecortin family. The protein contains two N-terminal doublecortin domains, which bind microtubules and regulate microtubule polymerization, a C-terminal serine/threonine protein kinase domain, which shows substantial homology to Ca2+/calmodulin-dependent protein kinase, and a serine/proline-rich domain in between the doublecortin and the protein kinase domains, which mediates multiple protein-protein interactions. The microtubule-polymerizing activity of the encoded protein is independent of its protein kinase activity. The protein is involved in several different cellular processes, including neuronal migration, retrograde transport, neuronal apoptosis and neurogenesis. This gene is up-regulated by brain-derived neurotrophic factor and associated with memory and general cognitive abilities.

L1CAM, or L1 cell adhesion molecule, also known as S10, HSAS, MASA, MIC5, SPG1, CAML1, CD171, HSAS1, N-CAML1, NCAM-L1, N-CAM-L1 is an axonal glycoprotein belonging to the immunoglobulin supergene family. The ectodomain, consisting of several immunoglobulin-like domains and fibronectin-like repeats (type III), is linked via a single transmembrane sequence to a conserved cytoplasmic domain. This cell adhesion molecule plays an important role in nervous system development, including neuronal migration and differentiation. Mutations in the gene cause X-linked neurological syndromes known as CRASH (corpus callosum hypoplasia, retardation, aphasia, spastic paraplegia and hydrocephalus).

PI4K, phosphatidylinositol 4-kinase A, also known as PIK4CA, PMGYCHA, Pi4K230, PI4K-ALPHA, catalyzes the first committed step in the biosynthesis of phosphatidylinositol 4,5-bisphosphate. The mammalian PI 4-kinases have been classified into two types, II and III, based on their molecular mass, and modulation by detergent and adenosine.

MYO5A, also named myosin V, also known as GS1, MYO5, MYH12, MYR12, is a protein associated with the centrosome and appear to be involved in cellular proliferation or in the polarized movement of the centrosome. Myosin V is a class of actin-based motor proteins involved in cytoplasmic vesicle transport and anchorage, spindle-pole alignment and mRNA translocation. The protein encoded by this gene is abundant in melanocytes and nerve cells. Mutations in the MYO5A gene is associated with the Griscelli syndrome, a rare autosomal recessive disorder characterized by pigmentary dilution and either central nervous system or immunologic defects.

ANK1, ankyrin 1, also known as ANK, SPH1, SPH2, is an integral membrane protein to the underlying spectrin-actin cytoskeleton and plays key roles in activities such as cell motility, activation, proliferation, contact and the maintenance of specialized membrane domains. This protein is composed of three structural domains: an amino-terminal domain containing multiple ankyrin repeats; a central region with a highly conserved spectrin binding domain; and a carboxy-terminal regulatory domain which is the least conserved and subject to variation. Ankyrin 1, was first discovered in the erythrocytes, but since has also been found in brain and muscles. Mutations in erythrocytic ankyrin 1 have been associated in approximately half of all patients with hereditary spherocytosis.

PURB, purine rich element binding protein B, also known as PURBETA, is a single-stranded DNA-binding protein. It binds preferentially to the single strand of the purine-rich element termed PUR, which is present at origins of replication and in gene flanking regions in a variety of eukaryotes from yeasts through humans. Thus, it is implicated in the control of both DNA replication and transcription. Deletion of this gene has been associated with myelodysplastic syndrome and acute myelogenous leukemia.

The amounts of the proteins disclosed herein were observed as being significantly increased or decreased, depending on the protein, in cerebral tissue from individuals suffering from a cerebral creatine deficiency syndrome, i.e., a creatine transporter (CRTR) deficiency, compared with healthy individuals.

An increased amount of a protein compared with a predetermined reference value may be an increase of about 1.3, or of about 1.4, or of about 1.5, or of about 1.6, or of about 1.7, or of about 1.8, or of about 1.9, or of about 2.0, or of about 2.5, or of about 3.0, or of about 4.0, or of about 5.0, or of about 6.0, or of about 7.0, or of about 8.0, or of about 9.0, or of about 10.0-fold.

In some embodiments, an increased amount of a protein compared with a predetermined reference value may be an increase of at least 1.5-fold.

A decreased amount of a protein compared with a predetermined reference value may be a decrease of about 1.1-fold, or of about 1.2, or of about 1.3, or of about 1.4, or of about 1.5, or of about 1.6, or of about 1.7, or of about 1.8, or of about 1.9, or of about 2.0, or of about 2.5, or of about 3.0, or of about 4.0, or of about 5.0, or of about 6.0, or of about 7.0, or of about 8.0, or of about 9.0, or of about 10.0-fold.

In some embodiments, a decrease amount of a protein compared with a predetermined reference value may be a decrease of at least 1.5-fold.

A predetermined reference value of a protein measured herein may be obtained from a healthy individual or a group of healthy individuals. A predetermined reference value may represent a mean of measures obtained from a group of healthy individual. To be comparable, the amounts of proteins measured from a biological sample and the predetermined reference values are obtained with same measurement methods. The methods are the same for the same protein but may differ for different proteins.

In some approaches, amounts of biomarkers may be processed into more valuable forms of information, e.g., by using either common mathematical transformations such as logarithmic or logistic functions. Other data processing approaches, such as normalization of biomarker results in reference to a population's mean values, etc. are also well known to those skilled in the art and can be used.

The amount of BDNF in a biological sample obtained from an individual suffering from a cerebral creatine deficiency syndrome, i.e., a creatine transporter (CRTR) deficiency is significantly increased compared with the amount of BDNF in a biological sample, e.g., blood, plasma or serum sample, obtained from a healthy individual or a predetermined reference value. For example, the decrease of the measured amount of BDNF in a biological sample obtained from an individual suffering from a cerebral creatine deficiency syndrome, for example a creatine transporter (CRTR) deficiency, may be at least 1.3, or at least 1.5 times the predetermined reference value.

The amount of KIF1A in a biological sample obtained from an individual suffering from a cerebral creatine deficiency syndrome, i.e., a creatine transporter (CRTR) deficiency is significantly increased compared with the amount of KIF1A in a biological sample obtained from a healthy individual or a predetermined reference value. For example, the increase of the measured amount of KIF1A in a biological sample obtained from an individual suffering from a cerebral creatine deficiency syndrome, for example a creatine transporter (CRTR) deficiency, may be at least 1.3, or at least 1.5 times the predetermined reference value.

The amount of MECP2 in a biological sample obtained from an individual suffering from a cerebral creatine deficiency syndrome, i.e., a creatine transporter (CRTR) deficiency is significantly decreased compared with the amount of MECP2 in a biological sample obtained from a healthy individual or a predetermined reference value. For example, the decrease of the measured amount of MECP2 in a biological sample obtained from an individual suffering from a cerebral creatine deficiency syndrome, for example a creatine transporter (CRTR) deficiency, may be at least 1.3, or at least 1.5 times the predetermined reference value.

The amount of PLCB1 in a biological sample obtained from an individual suffering from a cerebral creatine deficiency syndrome, i.e., a creatine transporter (CRTR) deficiency is significantly decreased compared with the amount of PLCB1 in a biological sample obtained from a healthy individual or a predetermined reference value. For example, the decrease of the measured amount of PLCB1 in a biological sample obtained from an individual suffering from a cerebral creatine deficiency syndrome, for example a creatine transporter (CRTR) deficiency, may be at least 1.3, or at least 1.5 times the predetermined reference value.

The measured amounts of MeCP2 and PLCB1 in the tested brain tissues were significantly downregulated in an animal model of a cerebral creatine deficiency syndrome, i.e., a creatine transporter (CRTR) deficiency, compared to healthy individuals, while the amount of KIF1A, was significantly upregulated. Further, the DCE treatment was able to restore comparable amounts of the proteins between the healthy and the Slc6a8^{-/y} model animals treated with DCE.

The amount of FABP7 in a biological sample obtained from an individual suffering from a cerebral creatine deficiency syndrome, i.e., a creatine transporter (CRTR) deficiency is significantly increased compared with the amount of FABP7 in a biological sample obtained from a healthy individual or a predetermined reference value. For example, the increase of the measured amount of FABP7 in a biological sample obtained from an individual suffering from a cerebral creatine deficiency syndrome, for example a creatine transporter (CRTR) deficiency, may be at least 1.3, or at least 1.5 times the predetermined reference value.

The amount of LMNB1 in a biological sample obtained from an individual suffering from a cerebral creatine deficiency syndrome, i.e., a creatine transporter (CRTR) deficiency is significantly increased compared with the amount of LMNB1 in a biological sample obtained from a healthy individual or a predetermined reference value. For example, the increase of the measured amount of LMNB1 in a biological sample obtained from an individual suffering from a cerebral creatine deficiency syndrome, for example a creatine transporter (CRTR) deficiency, may be at least 1.3, or at least 1.5 times the predetermined reference value.

The amount of IGSF8 in a biological sample obtained from an individual suffering from a cerebral creatine deficiency syndrome, i.e., a creatine transporter (CRTR) deficiency is significantly decreased compared with the amount of IGSF8 in a biological sample obtained from a healthy individual or a predetermined reference value. For example, the decrease of the measured amount of IGSF8 in a biological sample obtained from an individual suffering from a cerebral creatine deficiency syndrome, for example a creatine transporter (CRTR) deficiency, may be at least 1.3, or at least 1.5 times the predetermined reference value.

The measured amounts of FABP7 and LMNB1 in the tested brain tissues were significantly upregulated in an animal model of a cerebral creatine deficiency syndrome, i.e., a creatine transporter (CRTR) deficiency, compared to healthy individuals, while the amount of IGSF8 was significantly downregulated. Further, the DCE treatment was able to restore comparable amounts of the proteins between the healthy and the Slc6a8^{-/y} model animals treated with DCE.

The amount of NCAM1 in a biological sample obtained from an individual suffering from a cerebral creatine deficiency syndrome, i.e., a creatine transporter (CRTR) deficiency is significantly increased compared with the amount of NCAM1 in a biological sample obtained from a healthy individual or a predetermined reference value. For example, the increase of the measured amount of NCAM1 in a biological sample obtained from an individual suffering from a cerebral creatine deficiency syndrome, for example a creatine transporter (CRTR) deficiency, may be at least 1.3, or at least 1.5 times the predetermined reference value.

The amount of ANXA5 in a biological sample obtained from an individual suffering from a cerebral creatine deficiency syndrome, i.e., a creatine transporter (CRTR) deficiency is significantly decreased compared with the amount of ANXA5 in a biological sample obtained from a healthy individual or a predetermined reference value. For example, the decrease of the measured amount of ANXA5 in a biological sample obtained from an individual suffering from a cerebral creatine deficiency syndrome, for example a creatine transporter (CRTR) deficiency, may be at least 1.3, or at least 1.5 times the predetermined reference value.

The amount of DCLK1 in a biological sample obtained from an individual suffering from a cerebral creatine deficiency syndrome, i.e., a creatine transporter (CRTR) deficiency is significantly increased compared with the amount of DCLK1 in a biological sample obtained from a healthy individual or a predetermined reference value. For example, the increase of the measured amount of DCLK1 in a biological sample obtained from an individual suffering from a cerebral creatine deficiency syndrome, for example a creatine transporter (CRTR) deficiency, may be at least 1.3, or at least 1.5 times the predetermined reference value.

The amount of L1CAM in a biological sample obtained from an individual suffering from a cerebral creatine deficiency syndrome, i.e., a creatine transporter (CRTR) deficiency is significantly increased compared with the amount of L1CAM in a biological sample obtained from a healthy individual or a predetermined reference value. For example, the increase of the measured amount of L1CAM in a biological sample obtained from an individual suffering from a cerebral creatine deficiency syndrome, for example a creatine transporter (CRTR) deficiency, may be at least 1.3, or at least 1.5 times the predetermined reference value.

The amount of PI4KA in a biological sample obtained from an individual suffering from a cerebral creatine deficiency syndrome, i.e., a creatine transporter (CRTR) deficiency is significantly decreased compared with the amount of PI4KA in a biological sample obtained from a healthy individual or a predetermined reference value. For example, the decrease of the measured amount of PI4KA in a biological sample obtained from an individual suffering from a cerebral creatine deficiency syndrome, for example a creatine transporter (CRTR) deficiency, may be at least 1.3, or at least 1.5 times the predetermined reference value.

The amount of MYO5A in a biological sample obtained from an individual suffering from a cerebral creatine deficiency syndrome, i.e., a creatine transporter (CRTR) deficiency is significantly increased compared with the amount of MYO5A in a biological sample obtained from a healthy individual or a predetermined reference value. For example, the increase of the measured amount of MYO5A in a biological sample obtained from an individual suffering from a cerebral creatine deficiency syndrome, for example a creatine transporter (CRTR) deficiency, may be at least 1.3, or at least 1.5 times the predetermined reference value.

The amount of ANK1 in a biological sample obtained from an individual suffering from a cerebral creatine deficiency syndrome, i.e., a creatine transporter (CRTR) deficiency is significantly decreased compared with the amount of ANK1 in a biological sample obtained from a healthy individual or a predetermined reference value. For example, the decrease of the measured amount of ANK1 in a biological sample obtained from an individual suffering from a cerebral creatine deficiency syndrome, for example a creatine transporter (CRTR) deficiency, may be at least 1.3, or at least 1.5 times the predetermined reference value.

The amount of PURB in a biological sample obtained from an individual suffering from a cerebral creatine deficiency syndrome, i.e., a creatine transporter (CRTR) deficiency is significantly increased compared with the amount of PURB in a biological sample obtained from a healthy individual or a predetermined reference value. For example, the increase of the measured amount of PURB in a biological sample obtained from an individual suffering from a cerebral creatine deficiency syndrome, for example a creatine transporter (CRTR) deficiency, may be at least 1.3, or at least 1.5 times the predetermined reference value.

The measured amounts of NCAM1, DCLK1, L1CAM, MYO5 and PURB in the tested brain tissues were significantly upregulated in an animal model of a cerebral creatine deficiency syndrome, i.e., a creatine transporter (CRTR) deficiency, compared to healthy individuals, while the amount of ANXA5, PI4K-A and ANK1 were significantly downregulated. Further, the DCE treatment was able to restore comparable amounts of the proteins between the healthy and the Slc6a8^{-/y} model animals treated with DCE.

### Combinations of Proteins

The methods disclosed herein may use various combinations of the disclosed biomarkers. Combinations of some biomarkers may provide performance characteristics of the diagnosis that is superior to that of the individual biomarkers.

Various classification and statistical models can be applied to datasets comprising combinations of biomarkers to identify combinations having a correlation with the clinical characteristics of a cerebral creatine deficiency syndrome. These models are well known in the art, including, but are not limited to, Linear Model, Non-Linear Model, Linear DA, quadratic DA, Naive Bayes, Linear Regression, Quadratic Regression, KNN, Linear SVM, SVM with 2nd order polynomial Kernel, SVM with 3rd order polynomial Kernel, Neural Networks, Parzen Windows, Fuzzy Logic, and Decision Trees. In some embodiments, a multivariate statistical model using one-way ANOVA with Bonferroni post-hoc analysis comparison may be applied.

In some embodiments, diagnosis of a cerebral creatine deficiency syndrome may be made by calculating an index based on the combinations of two or more biomarkers. A reference value for an index may be determined by ROC analysis, comparing a healthy population versus a population with a cerebral creatine deficiency syndrome. A reference value can be derived from ROC analysis, selecting the reference value as that which maximizes sensitivity while keeping the specificity above a user-defined threshold. The reference value can also be selected as that which maximizes specificity while keeping the sensitivity above a user-defined threshold. A reference value may be selected as one such that the specificity is at the maximum when the user-defined threshold of sensitivity is 80% based on the ROC analysis.

In some embodiments, the biomarker to be used according to the present disclosure may be protein BDNF.

In some embodiments, the biomarker to be used according to the present disclosure may be at least one protein selected from BDNF, KIF1A, MeCP2, PLCB1, and a combination thereof.

In some embodiments, the biomarker to be used according to the present disclosure may be a set of proteins comprising or consisting of KIF1A, MeCP2, and PLCB1.

In some embodiments, the biomarker to be used according to the present disclosure may be a set of proteins comprising or consisting of KIF1A, MeCP2, PLCB1, and BDNF.

In another embodiment, the biomarker may be a set of proteins comprising or consisting of KIF1A, MeCP2, and PLCB1, and at least one protein from the group FABP7, LMNB1, and IGSF8, and combinations thereof.

In another embodiment, the biomarker may be a set of proteins comprising or consisting of KIF1A, MeCP2, PLCB1, and BDNF, and at least one protein from the group FABP7, LMNB1, and IGSF8, and combinations thereof.

In another embodiment, the biomarker may be a set of proteins comprising or consisting of KIF1A, MeCP2, PLCB1, and FABP7, and one of LMNB1 and IGSF8, and their combination.

In another embodiment, the biomarker may be a set of proteins comprising or consisting of KIF1A, MeCP2, PLCB1, BDNF, and FABP7, and one of LMNB1 and IGSF8, and their combination.

In another embodiment, the biomarker may be a set of proteins comprising or consisting of KIF1A, MeCP2, PLCB1, and LMNB1, and one of FABP7 and IGSF8, and their combination.

In another embodiment, the biomarker may be a set of proteins comprising or consisting of KIF1A, MeCP2, PLCB1, BDNF, and LMNB1, and one of FABP7 and IGSF8, and their combination.

In another embodiment, the biomarker may be a set of proteins comprising or consisting of KIF1A, MeCP2, PLCB1, and IGSF8, and one of FABP7 and LMNB1, and their combination.

In another embodiment, the biomarker may be a set of proteins comprising or consisting of KIF1A, MeCP2, PLCB1, BDNF, and IGSF8, and one of FABP7 and LMNB1, and their combination.

In another embodiment, the biomarker may be a set of proteins comprising or consisting of KIF1A, MeCP2, PLCB1, FABP7, LMNB1, and IGSF8.

In another embodiment, the biomarker may be a set of proteins comprising or consisting of KIF1A, MeCP2, PLCB1, BDNF, FABP7, LMNB1, and IGSF8.

In some embodiments, a biomarker disclosed herein may comprise any of the above indicated sets of proteins combined with any further proteins such as a protein from the group NCAM1, ANXA5, DCLK1, L1CAM, PI4K-A, MYO5, ANK1 and PURB, and combinations thereof.

In another embodiment, the biomarker may be a set of proteins comprising or consisting of KIF1A, MeCP2, and PLCB1, and at least one protein from the group NCAM1, ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, and BDNF, and at least one protein from the group NCAM1, ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, and NCAM1, and at least one protein from the group ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, BDNF, and NCAM1, and at least one protein from the group ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, and ANXA5, and at least one protein from the group NCAM1, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, BDNF, and ANXA5, and at least one protein from the group NCAM1, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, and DCLK1, and at least one protein from the group NCAM1, ANXA5, L1CAM, PI4K-A, MYO5, ANK1 and PURB, and combinations thereof.

In another embodiment, the biomarker may be a set of proteins comprising or consisting of KIF1A, MeCP2, PLCB1, BDNF, and DCLK1, and at least one protein from the group NCAM1, ANXA5, L1CAM, PI4K-A, MYO5, ANK1 and PURB, and combinations thereof.

In another embodiment, the biomarker may be a set of proteins comprising or consisting of KIF1A, MeCP2, PLCB1, and L1CAM, and at least one protein from the group NCAM1, ANXA5, DCLK1, PI4K-A, MYO5, ANK1 and PURB, and combinations thereof.

In another embodiment, the biomarker may be a set of proteins comprising or consisting of KIF1A, MeCP2, PLCB1, BDNF, and L1CAM, and at least one protein from the group NCAM1, ANXA5, DCLK1, PI4K-A, MYO5, ANK1 and PURB, and combinations thereof.

In another embodiment, the biomarker may be a set of proteins comprising or consisting of KIF1A, MeCP2, PLCB1, and PI4K-A, and at least one protein from the group NCAM1, ANXA5, DCLK1, L1CAM, MYO5, ANK1 and PURB, and combinations thereof.

In another embodiment, the biomarker may be a set of proteins comprising or consisting of KIF1A, MeCP2, PLCB1, BDNF, and PI4K-A, and at least one protein from the group NCAM1, ANXA5, DCLK1, L1CAM, MYO5, ANK1 and PURB, and combinations thereof.

In another embodiment, the biomarker may be a set of proteins comprising or consisting of KIF1A, MeCP2, PLCB1, and MYO5, and at least one protein from the group NCAM1, ANXA5, DCLK1, L1CAM, PI4K-A, ANK1 and PURB, and combinations thereof.

In another embodiment, the biomarker may be a set of proteins comprising or consisting of KIF1A, MeCP2, PLCB1, BDNF, and MYO5, and at least one protein from the group NCAM1, ANXA5, DCLK1, L1CAM, PI4K-A, ANK1 and PURB, and combinations thereof.

In another embodiment, the biomarker may be a set of proteins comprising or consisting of KIF1A, MeCP2, PLCB1, and ANK1, and at least one protein from the group NCAM1, ANXA5, DCLK1, L1CAM, PI4K-A, MYO5 and PURB, and combinations thereof.

In another embodiment, the biomarker may be a set of proteins comprising or consisting of KIF1A, MeCP2, PLCB1, BDNF, and ANK1, and at least one protein from the group NCAM1, ANXA5, DCLK1, L1CAM, PI4K-A, MYO5 and PURB, and combinations thereof.

In another embodiment, the biomarker may be a set of proteins comprising or consisting of KIF1A, MeCP2, PLCB1, and PURB, and at least one protein from the group NCAM1, ANXA5, DCLK1, L1CAM, PI4K-A, MYO5 and ANK1, and combinations thereof.

In another embodiment, the biomarker may be a set of proteins comprising or consisting of KIF1A, MeCP2, PLCB1, BDNF, and PURB, and at least one protein from the group NCAM1, ANXA5, DCLK1, L1CAM, PI4K-A, MYO5 and ANK1, and combinations thereof.

In some embodiments, a biomarker disclosed herein may comprise any of the above indicated sets of proteins combined with any further proteins such as a protein from the group FABP7, LMNB1, and IGSF8, and combinations thereof.

In another embodiment, the biomarker may be a set of proteins comprising or consisting of KIF1A, MeCP2, and PLCB1, and at least one protein from the group FABP7, LMNB1, IGSF8, NCAM1, ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, and BDNF, and at least one protein from the group FABP7, LMNB1, IGSF8, NCAM1, ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, FABP7, LMNB1, and IGSF8, and at least one protein from the group NCAM1, ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, BDNF, FABP7, LMNB1, and IGSF8, and at least one protein from the group NCAM1, ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, FABP7, LMNB1, IGSF8, and NCAM1, and at least one protein from the group ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, BDNF, FABP7, LMNB1, IGSF8, and NCAM1, and at least one protein from the group ANXA5, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, FABP7, LMNB1, and IGSF8, and ANXA5, and at least one protein from the group NCAM1, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, BDNF, FABP7, LMNB1, and IGSF8, and ANXA5, and at least one protein from the group NCAM1, DCLK1, L1CAM, PI4K-A, MYO5, ANK1 and PURB, and combinations thereof.

In another embodiment, the biomarker may be a set of proteins comprising or consisting of KIF1A, MeCP2, PLCB1, FABP7, LMNB1, IGSF8, and DCLK1, and at least one protein from the group NCAM1, ANXA5, L1CAM, PI4K-A, MYO5, ANK1 and PURB, and combinations thereof.

In another embodiment, the biomarker may be a set of proteins comprising or consisting of KIF1A, MeCP2, PLCB1, FABP7, LMNB1, IGSF8, and DCLK1, and at least one protein from the group NCAM1, ANXA5, L1CAM, PI4K-A, MYO5, ANK1 and PURB, and combinations thereof.

In another embodiment, the biomarker may be a set of proteins comprising or consisting of KIF1A, MeCP2, PLCB1, FABP7, LMNB1, IGSF8, and L1CAM, and at least one protein from the group NCAM1, ANXA5, DCLK1, PI4K-A, MYO5, ANK1 and PURB, and combinations thereof.

In another embodiment, the biomarker may be a set of proteins comprising or consisting of KIF1A, MeCP2, PLCB1, BDNF, FABP7, LMNB1, IGSF8, and L1CAM, and at least one protein from the group NCAM1, ANXA5, DCLK1, PI4K-A, MYO5, ANK1 and PURB, and combinations thereof.

In another embodiment, the biomarker may be a set of proteins comprising or consisting of KIF1A, MeCP2, PLCB1, FABP7, LMNB1, IGSF8, and PI4K-A, and at least one protein from the group NCAM1, ANXA5, DCLK1, L1CAM, MYO5, ANK1 and PURB, and combinations thereof.

In another embodiment, the biomarker may be a set of proteins comprising or consisting of KIF1A, MeCP2, PLCB1, BDNF, FABP7, LMNB1, IGSF8, and PI4K-A, and at least one protein from the group NCAM1, ANXA5, DCLK1, L1CAM, MYO5, ANK1 and PURB, and combinations thereof.

In another embodiment, the biomarker may be a set of proteins comprising or consisting of KIF1A, MeCP2, PLCB1, FABP7, LMNB1, IGSF8, and MYO5, and at least one protein from the group NCAM1, ANXA5, DCLK1, L1CAM, PI4K-A, ANK1 and PURB, and combinations thereof.

In another embodiment, the biomarker may be a set of proteins comprising or consisting of KIF1A, MeCP2, PLCB1, BDNF, FABP7, LMNB1, IGSF8, and MYO5, and at least one protein from the group NCAM1, ANXA5, DCLK1, L1CAM, PI4K-A, ANK1 and PURB, and combinations thereof.

In another embodiment, the biomarker may be a set of proteins comprising or consisting of KIF1A, MeCP2, PLCB1, FABP7, LMNB1, IGSF8, and ANK1, and at least one protein from the group NCAM1, ANXA5, DCLK1, L1CAM, PI4K-A, MYO5 and PURB, and combinations thereof.

In another embodiment, the biomarker may be a set of proteins comprising or consisting of KIF1A, MeCP2, PLCB1, BDNF, FABP7, LMNB1, IGSF8, and ANK1, and at least one protein from the group NCAM1, ANXA5, DCLK1, L1CAM, PI4K-A, MYO5 and PURB, and combinations thereof.

In another embodiment, the biomarker may be a set of proteins comprising or consisting of KIF1A, MeCP2, PLCB1, FABP7, LMNB1, IGSF8, and PURB, and at least one protein from the group NCAM1, ANXA5, DCLK1, L1CAM, PI4K-A, MYO5 and ANK1, and combinations thereof.

In another embodiment, the biomarker may be a set of proteins comprising or consisting of KIF1A, MeCP2, PLCB1, BDNF, FABP7, LMNB1, IGSF8, and PURB, and at least one protein from the group NCAM1, ANXA5, DCLK1, L1CAM, PI4K-A, MYO5 and ANK1, and combinations thereof.

In another embodiment, the biomarker may be a set of proteins comprising or consisting of KIF1A, MeCP2, PLCB1, NCAM1, ANXA5, DCLK1, L1CAM, PI4K-A, MYO5, ANK1 and PURB and at least one protein from the group FABP7, LMNB1, IGSF8, and combinations thereof.

In another embodiment, the biomarker may be a set of proteins comprising or consisting of KIF1A, MeCP2, PLCB1, BDNF, NCAM1, ANXA5, DCLK1, L1CAM, PI4K-A, MYO5, ANK1 and PURB and at least one protein from the group FABP7, LMNB1, IGSF8, and combinations thereof.

In another embodiment, the biomarker may be a set of proteins comprising or consisting of KIF1A, MeCP2, PLCB1, NCAM1, ANXA5, DCLK1, L1CAM, PI4K-A, MYO5, ANK1, PURB, and FABP7, and one of LMNB1 and IGSF8, and their combination.

In another embodiment, the biomarker may be a set of proteins comprising or consisting of KIF1A, MeCP2, PLCB1, BDNF, NCAM1, ANXA5, DCLK1, L1CAM, PI4K-A, MYO5, ANK1, PURB, and FABP7, and one of LMNB1 and IGSF8, and their combination.

In another embodiment, the biomarker may be a set of proteins comprising or consisting of KIF1A, MeCP2, PLCB1, NCAM1, ANXA5, DCLK1, L1CAM, PI4K-A, MYO5, ANK1, PURB, and LMNB1, and one of FABP7 and IGSF8, and their combination.

In another embodiment, the biomarker may be a set of proteins comprising or consisting of KIF1A, MeCP2, PLCB1, BDNF, NCAM1, ANXA5, DCLK1, L1CAM, PI4K-A, MYO5, ANK1, PURB, and LMNB1, and one of FABP7 and IGSF8, and their combination.

In another embodiment, the biomarker may be a set of proteins comprising or consisting of KIF1A, MeCP2, PLCB1, NCAM1, ANXA5, DCLK1, L1CAM, PI4K-A, MYO5, ANK1, PURB, and IGSF8, and one of FABP7 and LMNB1, and their combination.

In another embodiment, the biomarker may be a set of proteins comprising or consisting of KIF1A, MeCP2, PLCB1, NCAM1, ANXA5, DCLK1, L1CAM, PI4K-A, MYO5, ANK1, PURB, and IGSF8, and one of FABP7 and LMNB1, and their combination.

In another embodiment, the biomarker may be a set of proteins comprising or consisting of KIF1A, MeCP2, PLCB1, FABP7, LMNB1, and IGSF8, ANK1, NCAM1, ANXA5, DCLK1, L1CAM, PI4K-A, MYO5 and PURB.

In another embodiment, the biomarker may be a set of proteins comprising or consisting of KIF1A, MeCP2, PLCB1, BDNF, FABP7, LMNB1, and IGSF8, ANK1, NCAM1, ANXA5, DCLK1, L1CAM, PI4K-A, MYO5 and PURB.

### Dosage of the Proteins

In some embodiments, the step of measuring an amount of a protein may be performed by using one or more techniques selected from the group consisting of gas chromatography, liquid chromatography, mass spectrometry, immunoassay, ELISA, enzymatic or biochemical reactions and NMR, or combinations of two or more of these methods.

The measured amount of a protein in a biological sample can be compared directly with the measured amount of the same protein of a healthy reference individual, i.e., a predetermined value of reference, such as comparing the concentration of the protein present in the biological sample with the concentration of the same protein in a corresponding healthy reference sample. Both amounts are preferably measured with the same technique to avoid discrepancies caused by the use of different techniques.

In some embodiments, a mass-spectrometry may be used to determine the presence or absence of a measured protein in a sample.

A measure by mass-spectrometry has the advantages that determining the amount of a protein can be realized with high precision and sensitivity. Further, such embodiment has the advantages that this technique is able to measure a huge number of proteins in a sample; in other words, with this technique a user might be able to measure the amount from lots of different proteins in just one run. Furthermore, mass spectrometry has the advantage that the detection of proteins can be realized from micro liter quantities of the biological sample.

As example of mass-spectrometry technique which may be used, one may refer to MS/MS technique. In such embodiment, peptide tolerance, MS/MS fragment tolerance, and a maximum of missed cleavages may be set at 5 ppm, 0.02 Da and 2, respectively. Carbamidomethylation of cysteine may be considered as fixed modification. Oxidation of methionine may be taken into account as variable modification. Peptides identified at a p-value≤0.05 in homology threshold mode and proteins identified with at least two distinct peptides may be selected (false discovery rate below 1%).

In some embodiments, an immunoassay of a sample may be used to obtain an absolute or relative amount or concentration of a measured protein in a sample. A measure by immunoassay has the advantage that this technique is quite cheap in building, but highly sensitive in measuring the amount of at least one protein.

Various well-known immunological methods may be used to specifically identify and/or quantify the disclosed proteins. These methods include, but are not limited to, immunologic- or antibody-based techniques include enzyme-linked immunosorbent assay (ELISA), radioimmunoassay (RIA), western blotting, immunofluorescence, microarrays, some chromatographic techniques (i.e., immunoaffinity chromatography), flow cytometry, immunoprecipitation. These methods are based on the specificity of an antibody or antibodies for a particular epitope or combination of epitopes associated with the protein of interest.

Methods of producing antibodies for use in protein or antibody arrays, or other immunology based assays for detection of the proteins disclosed herein are known in the art. For preparation of monoclonal antibodies directed towards a protein, any technique that provides for the production of antibody molecules by continuous cell lines in culture may be used. Such techniques include, but are not limited to, the hybridoma technique originally developed by Kohler and Milstein Nature (1975) 256:495-497, the trioma technique (Gustafsson et al., Hum. Antibodies Hybridomas (1991) 2:26-32), the human B-cell hybridoma technique (Kozbor et al., Immunology Today (1983) 4:72), and the EBV hybridoma technique to produce human monoclonal antibodies (Cole et al., In: Monoclonal Antibodies and Cancer Therapy, Alan R. Liss, Inc., (1985) 77-96). In addition, techniques described for the production of single chain antibodies (U.S. Pat. No. 4,946,778) can be adapted to produce a protein-specific antibodies. An additional embodiment of may utilize the techniques described for the construction of Fab expression libraries (Huse et al., Science (1989) 246:1275-1281) to allow rapid and easy identification of monoclonal Fab fragments with the desired specificity for a protein. Non-human antibodies can be “humanized” by known methods (e.g., U.S. Pat. No. 5,225,539).

Antibody fragments that contain the idiotypes of a protein can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments. Synthetic antibodies, e.g., antibodies produced by chemical synthesis, are useful in the present disclosure.

Antibodies or fragments thereof used in methods for measuring amounts of proteins may bear a reporter molecule. Numerous labels or reporter molecules may be used, such as:
(a) Radioisotopes, such as 35S, 14C, 125I, 3H, and 131I. Radioactivity can be measured using scintillation counting. Other radionuclides include 99Tc, 90Y, 111In, 32P, 11C, 15O, 13N, 18F, 51Cr, 57To, 226Ra, 60Co, 59Fe, 57Se, 152Eu, 67CU, 217Ci, 211At, 212Pb, 47Sc, 109Pd, 234Th, and 40K, 157Gd, 55Mn, 52Tr, and 56Fe.
(b) Colloidal gold particles.
(c) Fluorescent or chemiluminescent labels including, but not limited to, rare earth chelates (europium chelates), fluorescein and its derivatives, rhodamine and its derivatives, isothiocyanate, phycocyanin, allophycocyanin, phycoerythrin, o-phthaladehyde, fluorescamine, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, Texas Red, dansyl, Lissamine, umbelliferone, phycocrytherin, phycocyanin, or commercially available fluorophores such SPECTRUM ORANGE® and SPECTRUM GREEN® and/or derivatives of any one or more of the above. The fluorescent labels can be conjugated to the antibody using the techniques disclosed in Current Protocols in Immunology, supra, for example. Fluorescence can be quantified using a fluorimeter.
(d) Enzyme catalyzing a substrate-based colorimetric reaction. Various enzyme-substrate labels are available. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor.

Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Numerous enzyme-substrate combinations are available to those skilled in the art. Examples of enzyme-substrate combinations are:
(i) Horseradish peroxidase (HRP) with hydrogen peroxidase as a substrate, wherein the hydrogen peroxidase oxidizes a dye precursor, such as, e.g., 3,3′ diamino benzidine (DAB), which produces a brown end product; 3-amino-9-ethylcarbazole (AEC), which upon oxidation forms a rose-red end product; 4-chloro-1-napthol (CN), which precipitates as a blue end product; and p-Phenylenediamine dihydrochloride/pyrocatecol, which generates a blue-black product; orthophenylene diamine (OPD) and 3,3′,5,5′-tetramethyl benzidine hydrochloride (TMB);
(ii) alkaline phosphatase (AP) and para-Nitrophenyl phosphate, naphthol AS-MX phosphate, Fast Red TR and Fast Blue BB, napthol AS-BI phosphate, napthol AS-TR phosphate, 5-bromo-4-chloro-3-indoxyl phosphate (BCIP), Fast Red LB, Fast Garnet GBC, Nitro Blue Tetrazolium (NBT), and iodonitrotetrazolium violet (INT); and
(iii) 3-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

In one embodiment, the label or reporter molecule may be selected in the group consisting of a fluorescent molecule, a radioisotope, an enzyme, a biotin, a streptavidin. For example, a label may be a fluorescent molecule.

In some embodiments, a protein may be measured by a chemiluminescent immunoassay. For example, a sample is added to a reaction vessel coated with a monoclonal anti-protein, blocking reagent. After incubation in a reaction vessel, unbound materials are washed away. Then, an enzyme able to catalyze a colorimetric reaction, such as an alkaline phosphatase, conjugated to an anti-protein antibody is added to the vessel with a chemiluminescent substrate and light generated by the reaction is measured with a luminometer. The light production is directly proportional to the level of protein in the sample. The amount of protein in the sample is determined from a stored, multi-point calibration curve.

In some embodiments, a protein may be measured by an immunofluorescent assay. For example, a sample is added to a reaction vessel along coated with a monoclonal anti-protein and blocking reagent. After incubation in a reaction vessel, unbound materials are washed away. Then, an anti-protein antibody conjugated to a fluorescence label is added to the vessel and a fluorescent signal is measured with a fluorometer. The fluorescent signal is directly proportional to the amount of protein in the sample. The amount of protein in the sample is determined from a stored, multi-point calibration curve.

Numerous variations of those chemiluminescent and immunofluorescent assays are known in the art.

Further, non-immunological methods, based on the physical or chemical properties of the proteins, can also be used to measure the disclosed proteins. Numerous methods are well known in the art and can be used to analyze/detect products of various reactions involving a protein disclosed herein. The reaction products can be detected by means of fluorescence, luminescence, mass measurement, or electrophoresis, etc. Furthermore, reactions can occur in solution or on a solid support such as a glass slide, a chip, a bead, or the like.

In some embodiments, a dosage of a protein may be carried in a biological sample. A biological sample may be blood, plasma, serum, cerebrospinal fluid, or is a brain organoid prepared by dedifferentiation and reprogramming of fibroblast cells obtained from said individual.

A biological sample may be obtained from an individual or from a biological model. A biological model may be a Slc6a8^{-/y} mouse, obtained as disclosed in the Examples section or in Raffaele, M. et al. Novel translational phenotypes and biomarkers for creatine transporter deficiency. Brain Commun., (2020)).

A biological model may also be cultured cells isolated from an individual suffering from a cerebral creatine deficiency syndrome. Such cells may be used in primary culture or after transformation of the cells in a cell line. For example, suitable cells can be fibroblast cells, for example isolated from the skin or from a muscle of an individual suffering from a cerebral creatine deficiency syndrome.

Also, a biological model may be a brain organoid obtained by dedifferentiation and reprogramming of fibroblast cells isolated from an individual suffering from a cerebral creatine deficiency syndrome. Numerous methods are available in the art for preparing brain organoids. For example, one may mention the protocols described in Lancaster et al. (Generation of cerebral organoids from human pluripotent stem cells. Nat Protoc. 2014; 9(10), Nassor et al. (Long Term Gene Expression in Human Induced Pluripotent Stem Cells and Cerebral Organoids to Model a Neurodegenerative Disease. Front Cell Neurosci. 2020; 14:14) or in Pavoni et al. (Small-molecule induction of Aβ-42 peptide production in human cerebral organoids to model Alzheimer's disease associated phenotypes. PLoS One. 2018; 13(12):e0209150).

For example, brain organoids may be obtained starting from patient's primary fibroblasts reprogrammed, for example using the Sendai virus reprogramming method, into iPSC. The obtained iPSCs may be then differentiated in embryonic bodies, which may be then matured in primitive neuroepithelia, and then in brain organoids.

### Kits

An object of the present disclosure relates to a kit for diagnosing or aiding in diagnosing a cerebral creatine deficiency syndrome in an individual in need thereof, said kit comprising means for measuring an amount of at least one protein selected from BDNF, KIF1A, MeCP2, PLCB1, and a combination thereof, in an isolated biological sample.

In an embodiment, the kit comprises means for measuring an amount of the protein BDNF.

In some embodiments, the kit may comprise means for measuring an amount of each protein KIF1A, MeCP2, and PLCB1. In some embodiments, the kit may further comprise means for measuring an amount of the protein BDNF.

An object of the present disclosure relates to a kit for diagnosing or aiding in diagnosing a cerebral creatine deficiency syndrome in an individual in need thereof, said kit comprising means for measuring an amounts of each protein KIF1A, MeCP2, and PLCB1 in an isolated biological sample obtained from said individual.

In some embodiments, the kit may further comprise means for measuring an amount of the protein BDNF.

A kit may further comprise means for measuring amounts of at least one protein selected among FABP7, LMNB1, and IGSF8 and combinations thereof, and/or NCAM1, ANXA5, DCLK1, L1CAM, PI4K-A, MYO5, ANK1 and PURB, and combinations thereof.

According to another of its objects, the present disclosure relates to a use of a kit according to the disclosure for diagnosing a cerebral creatine deficiency syndrome.

The kit may be arranged in a way that the biological sample just has to be inserted into the kit, while a method disclosed herein may be carried out fully automatically. Therefore, an unskilled person may be able to use the kit.

The kit may include means which are necessary for determining the amounts of proteins used as biomarker for metabolic disease. This might be chromatographic means, spectrometric means, means for enzymatic or biochemical reactions, means for immunoassay, etc., as well as means for stabilizing the biological sample. The kit might not be limited in just one technical means for determining the amount of a biomarker. For example, the kit might have means for performing a HPLC (high performance liquid chromatography) or UHPLC (ultra-high performance liquid chromatography) coupled to a mass spectrometry or coupled to an immunoassay.

The kit may also include reagents which might be necessary for performing the methods disclosed herein. The reagents may be provided in any suitable form. The reagents might be stabilization reagents, buffer reagents, solvents etc.

In some embodiments, a kit disclosed herein may comprise immunoassay means for determining an amount of a biomarker. In this embodiment, an immunoassay might be an ELISA. Therefore, a kit may comprise separate compartments, tubes, valves and the like. A kit may also include reagents which are necessary for performing an immunoassay, like antibodies, buffers, blocking agents, detection reagents and the like. These reagents are well known in the art. For example, a kit may comprise antibodies or fragments thereof, specific for the proteins markers (primary antibodies), along with one or more secondary antibodies that may incorporate a detectable label; such antibodies may be used in an assay such as an ELISA. Alternately, the antibodies or fragments thereof may be fixed to a solid surface, e.g., an antibody array. The kit may contain a detectable label such as fluorescein, green fluorescent protein, rhodamine, cyanine dyes, Alexa dyes, luciferase, radiolabels, among others.

In some embodiments, a kit disclosed herein may comprise a mass-spectrometer as means for determining an amount of a protein.

The kit as disclosed herein may further comprise an instruction to measure an amount of at least one protein KIF1A, MeCP2, PLCB1, and BDNF, and a combination thereof.

The kit as disclosed herein may further comprise an instruction to measure at least the amount of the protein BDNF.

The kit as disclosed herein may comprise an instruction to measure amounts of each protein KIF1A, MeCP2, and PLCB1. The kit as disclosed herein may further comprise an instruction to measure an amount of the protein BDNF.

The kit as disclosed herein may further comprise an instruction to measure amounts of each protein KIF1A, MeCP2, and PLCB1, and optionally at least one protein selected among FABP7, LMNB1, and IGSF8 and combinations thereof, and/or NCAM1, ANXA5, DCLK1, L1CAM, PI4K-A, MYO5, ANK1 and PURB, and combinations thereof.

The kit as disclosed herein may further comprise an instruction to measure amounts of each protein KIF1A, MeCP2, PLCB1, and BDNF, and optionally at least one protein selected among FABP7, LMNB1, and IGSF8 and combinations thereof, and/or NCAM1, ANXA5, DCLK1, L1CAM, PI4K-A, MYO5, ANK1 and PURB, and combinations thereof.

The kit may comprise an instruction to compare the measured amounts of the proteins with predetermined reference values.

A kit of the disclosure may comprise one or more other containers, containing for example, wash reagents or buffers.

A kit may comprise means for acquiring a quantity of a biological sample, such as a blood or serum sample; wash reagents and buffers and means to detect and quantify the proteins as disclosed herein.

According to another of its objects, the disclosure relates to a use of a kit as disclosed herein in the methods disclosed herein.

### Uses

The uses of the disclosure may be in vivo or in vitro. In some embodiments the uses may be in vitro.

In one of its objects, the disclosure relates to a use of at least one protein selected from KIF1A, MeCP2, PLCB1, BDNF, and a combination thereof, as a biomarker of a cerebral creatine deficiency syndrome.

In one of its objects, the disclosure relates to a use of at least the protein BDNF as a biomarker of a cerebral creatine deficiency syndrome.

In one of its objects, the disclosure relates to a use of a set of proteins comprising KIF1A, MeCP2, and PLCB1 as a biomarker of a cerebral creatine deficiency syndrome.

Uses or biomarkers disclosed herein may further comprise a use of the protein BDNF.

Uses or biomarkers disclosed herein may further comprise a use of a protein selected among FABP7, LMNB1, and IGSF8 and combinations thereof.

Uses or biomarkers disclosed herein may further comprise a use of a protein selected from NCAM1, ANXA5, DCLK1, L1CAM, PI4K-A, MYO5, ANK1 and PURB, and combinations thereof.

In some embodiments, uses or biomarkers disclosed herein may further comprise a use of a protein selected from FABP7, LMNB1, IGSF8, NCAM1, ANXA5, DCLK1, L1CAM, PI4K-A, MYO5, ANK1 and PURB, and combinations thereof.

Uses or biomarkers disclosed herein may further comprise a use of FABP7, LMNB1, IGSF8, NCAM1, ANXA5, DCLK1, L1CAM, PI4K-A, MYO5, ANK1 and PURB.

Uses or biomarkers disclosed herein may comprise any combinations of proteins as disclosed herein.

A cerebral creatine deficiency syndrome considered herein may be a creatine transporter (CRTR) deficiency.

The biomarkers disclosed herein may be for use in methods for diagnosing or aiding in diagnosing a cerebral creatine deficiency syndrome.

The biomarkers disclosed herein may be for use in methods for monitoring a therapeutic efficacy of a candidate therapeutic agent proposed for preventing and/or treating a cerebral creatine deficiency syndrome.

The biomarkers disclosed herein may be for use in methods for monitoring an evolution of a cerebral creatine deficiency syndrome.

The biomarkers disclosed herein may be for use in methods for selecting a candidate therapeutic agent susceptible to be used for preventing and or treating a cerebral creatine deficiency syndrome.

The biomarkers disclosed herein may be for use in methods for manufacturing a diagnostic tool for diagnosing or aiding in diagnosing a cerebral creatine deficiency syndrome.

The cerebral creatine deficiency syndrome may be a creatine transporter (CRTR) deficiency.

In one of its objects, the disclosure relates to a biomarker for use in methods disclosed herein, wherein the biomarker is at least one protein selected from KIF1A, MeCP2, PLCB1, BDNF, and a combination thereof.

In one of its objects, the disclosure relates to a biomarker for use in methods disclosed herein, wherein the biomarker is at least the protein BDNF.

In one of its objects, the disclosure relates to a biomarker for use in methods disclosed herein, wherein the biomarker is of a set of proteins comprising KIF1A, MeCP2, and PLCB1.

In one of its objects, the disclosure relates to a biomarker for use in methods disclosed herein, wherein the biomarker further comprises the protein BDNF.

In one of its objects, the disclosure relates to a biomarker for use in a method for diagnosing a cerebral creatine deficiency syndrome in an individual in need thereof, wherein the biomarker is at least one protein selected from KIF1A, MeCP2, PLCB1, BDNF, and a combination thereof.

In one of its objects, the disclosure relates to a biomarker for use in methods disclosed herein, wherein the biomarker is at least the protein BDNF.

In one of its objects, the disclosure relates to a biomarker for use in a method for diagnosing a cerebral creatine deficiency syndrome in an individual in need thereof, wherein the biomarker is of a set of proteins comprising KIF1A, MeCP2, and PLCB1.

In one of its objects, the disclosure relates to a biomarker for use in methods disclosed herein, wherein the biomarker further comprises the protein BDNF.

A biomarker for use as disclosed herein may further comprise a protein selected from the group FABP7, LMNB1, and IGSF8, and combinations thereof.

A biomarker for use as disclosed herein may further comprise a use of a protein selected from the group NCAM1, ANXA5, DCLK1, L1CAM, PI4K-A, MYO5, ANK1 and PURB, and combinations thereof.

A biomarker for use as disclosed herein any combinations of proteins as disclosed herein.

A biomarker for use as disclosed herein may further comprise a use of FABP7, LMNB1, IGSF8, NCAM1, ANXA5, DCLK1, L1CAM, PI4K-A, MYO5, ANK1 and PURB

### Methods

The methods of the disclosure may be in vivo or in vitro. In some embodiments, the methods of the disclosure may be in vitro methods.

The methods of the disclosure may be for diagnosing or for diagnosing and treating a cerebral creatine deficiency syndrome, or for monitoring a therapeutic efficacy of a candidate therapeutic agent proposed for preventing and/or treating a cerebral creatine deficiency syndrome, or for selecting a candidate therapeutic agent for preventing and/or treating a cerebral creatine deficiency syndrome, or for monitoring an evolution of a cerebral creatine deficiency syndrome.

A cerebral creatine deficiency syndrome considered herein may be a creatine transporter (CRTR) deficiency.

One objects of the disclosure relates to a method for diagnosing a cerebral creatine deficiency syndrome in an individual in need thereof, said method comprising at least the steps of:
a) measuring an amount of at least one protein selected from BDNF, KIF1A, MeCP2, PLCB1, and a combination thereof, in an isolated biological sample obtained from said individual,
b) comparing the amount measured at step a) with a predetermined reference value,
wherein a difference between the measured amount and the predetermined reference value is indicative of a cerebral creatine deficiency syndrome in said individual.

In some embodiments, step a) comprises measuring an amount of the protein BDNF.

In some embodiments, step a) comprises measuring an amount of each protein KIF1A, MeCP2 and PLCB1. In some embodiments, step a) comprises further measuring an amount of the protein BDNF.

One objects of the disclosure relates to a method for diagnosing a cerebral creatine deficiency syndrome in an individual in need thereof, said method comprising the steps of:
a) measuring an amount of each protein KIF1A, MeCP2, and PLCB1 in an isolated biological sample obtained from said individual,
b) comparing each amount measured at step a) with a predetermined reference value,
wherein a difference observed at step b) between the measured amounts and the predetermined reference values is indicative of a cerebral creatine deficiency syndrome in said individual.

In some embodiments, a method disclosed herein may further comprise at step a) a measure of a further protein or a combination of proteins as above described. In such embodiments, the method further comprises the comparison of the measured amounts of protein with further corresponding reference values.

In some embodiments, step a) comprises further measuring an amount of the protein BDNF.

In some embodiments, a method may further comprise at step a) a measure of an amount of at least one protein from the group FABP7, LMNB1, and IGSF8, and combinations thereof.

In some embodiments, the method may further comprise at step a) a measure of an amount of at least one protein from the group NCAM1, ANXA5, DCLK1, L1CAM, PI4K-A, MYO5, ANK1 and PURB, and combinations thereof.

In some embodiments, the method may further comprise a measure of an amount of at least one protein from the group FABP7, LMNB1, IGSF8, NCAM1, ANXA5, DCLK1, L1CAM, PI4K-A, MYO5, ANK1 and PURB, and combinations thereof.

In some embodiments, the method may further comprise a measure of an amount of FABP7, LMNB1, IGSF8, NCAM1, ANXA5, DCLK1, L1CAM, PI4K-A, MYO5, ANK1 and PURB.

An observed increase of the amount of BDNF compared to a reference value may be indicative of a cerebral creatine deficiency syndrome.

An observed increase of the amount of KIF1A compared to a reference value may be indicative of a cerebral creatine deficiency syndrome.

An observed decrease of the amount of MECP2 compared to a reference value may be indicative of a cerebral creatine deficiency syndrome.

An observed decrease of the amount of PLCB1 compared to a reference value may be indicative of a cerebral creatine deficiency syndrome.

An observed increase of the amount of FABP7 compared to a reference value may be indicative of a cerebral creatine deficiency syndrome.

An observed increase of the amount of LMNB1 compared to a reference value may be indicative of a cerebral creatine deficiency syndrome.

An observed decrease of the amount of IGSF8 compared to a reference value may be indicative of a cerebral creatine deficiency syndrome.

An observed increase of the amount of NCAM1 compared to a reference value may be indicative of a cerebral creatine deficiency syndrome.

An observed decrease of the amount of ANXA5 compared to a reference value may be indicative of a cerebral creatine deficiency syndrome.

An observed increase of the amount of DCLK1 compared to a reference value may be indicative of a cerebral creatine deficiency syndrome.

An observed increase of the amount of L1CAM compared to a reference value may be indicative of a cerebral creatine deficiency syndrome.

An observed decrease of the amount of PI4K-A compared to a reference value may be indicative of a cerebral creatine deficiency syndrome.

An observed increase of the amount of MYO5 compared to a reference value may be indicative of a cerebral creatine deficiency syndrome.

An observed decrease of the amount of ANK1 compared to a reference value may be indicative of a cerebral creatine deficiency syndrome.

An observed increase of the amount of PURB compared to a reference value may be indicative of a cerebral creatine deficiency syndrome.

In some embodiments, an observed decrease of the amounts of MeCP2 and PLCB1 compared to reference values and an observed increase of the amount of KIF1A compared to a reference value may be indicative of a cerebral creatine deficiency syndrome.

In some embodiments, an observed decrease of the amounts of MeCP2 and PLCB1 compared to reference values and an observed increase of the amounts of KIF1A and BDNF compared to reference values may be indicative of a cerebral creatine deficiency syndrome.

In some embodiments, an observed increase of the amounts of FABP7 and LMNB1 compared to reference values and an observed decrease of the amount of IGSF8 compared to a reference value may be indicative of a cerebral creatine deficiency syndrome.

In some embodiments, an observed increase of the amounts of NCAM1, DCLK1, L1CAM, MYO5 and PURB compared to reference values and an observed decrease of the amounts of ANXA5, PI4K-A and ANK1 compared to reference values may be indicative of a cerebral creatine deficiency syndrome.

In some embodiments, an observed decrease of the amounts of MeCP2, PLCB1, IGSF8, ANXA5, PI4K-A and ANK1 compared to reference values and an observed increase of the amounts of KIF1A, FABP7, LMNB1, NCAM1, DCLK1, L1CAM, MYO5 and PURB compared to reference values may be indicative of a cerebral creatine deficiency syndrome.

In some embodiments, an observed decrease of the amounts of MeCP2, PLCB1, IGSF8, ANXA5, PI4K-A and ANK1 compared to reference values and an observed increase of the amounts of BDNF, KIF1A, FABP7, LMNB1, NCAM1, DCLK1, L1CAM, MYO5 and PURB compared to reference values may be indicative of a cerebral creatine deficiency syndrome.

One objects of the disclosure relates to a method for monitoring a therapeutic efficacy of a therapeutic treatment proposed for preventing and/or treating a cerebral creatine deficiency syndrome in an individual in need thereof, said method comprising the steps of:
a) measuring an amount of at least one protein selected from BDNF, KIF1A, MeCP2, PLCB1, and a combination thereof, in an isolated biological sample obtained from said individual before administration of said therapeutic treatment,
b) measuring an amount of at least one protein selected from BDNF, KIF1A, MeCP2, PLCB1, and a combination thereof, in an isolated biological sample obtained from said individual after administration of said therapeutic treatment, the protein or combination of proteins of step a) and b) being the same,
c) comparing the amounts measured at step a) with the amounts measured at step b),
wherein a difference between the measured amounts at step a) and at step b) is indicative of a therapeutic efficacy of said therapeutic treatment on said cerebral creatine deficiency syndrome.

In some embodiments, steps a) and b) comprise measuring an amount of the protein BDNF.

In some embodiments, steps a) and b) comprise measuring an amount of each protein KIF1A, MeCP2 and PLCB1. In some embodiments, steps a) and b) comprise further measuring an amount of the protein BDNF.

One objects of the disclosure relates to a method for monitoring a therapeutic efficacy of a therapeutic treatment proposed for preventing and/or treating a condition, the condition being a cerebral creatine deficiency syndrome in an individual in need thereof, said method comprising the steps of:
a) measuring an amount of each protein KIF1A, MeCP2, and PLCB1 in an isolated biological sample obtained from said individual before administration of said therapeutic treatment,
b) measuring an amount of each protein KIF1A, MeCP2, and PLCB1 in an isolated biological sample obtained from said individual after administration of said candidate therapeutic agent,
c) comparing the amounts measured at step a) with the amounts measured at step b),
wherein an observed difference between the measured amounts at step a) and at step b) is indicative of a therapeutic efficacy of said therapeutic treatment on said cerebral creatine deficiency syndrome.

In some embodiments, steps a) and b) comprise further measuring an amount of the protein BDNF.

A therapeutic treatment agent may be a behavioral treatment or an administration of a therapeutic agent, such as dodecyl creatine ester (DCE).

Suitable treatment for an individual diagnosed with a cerebral creatine deficiency syndrome, such a CTD, may include occupational, speech, and physical therapies to treat developmental disabilities and behavioral therapy to address behavior problems.

Suitable treatments with therapeutic agents which may be implemented include creatine monohydrate, L-arginine, glycine, dodecyl creatine ester, creatine analog such as cyclocreatine, and combinations thereof.

In some embodiments, a therapeutic agent may be dodecyl creatine ester.

In some embodiments, a therapeutic agent may be dodecyl creatine ester incorporated into lipid nanocapsules.

In some embodiments, the method may further comprise a measure of a further protein or a combination of proteins as above described.

In some embodiments, the method may further comprise a measure of an amount of at least one protein from the group FABP7, LMNB1, and IGSF8, and combinations thereof.

In some embodiments, the method may further comprise a measure of an amount of at least one protein from the group NCAM1, ANXA5, DCLK1, L1CAM, PI4K-A, MYO5, ANK1 and PURB, and combinations thereof.

In some embodiments, the method may further comprise a measure of an amount of at least one protein from the group FABP7, LMNB1, IGSF8, NCAM1, ANXA5, DCLK1, L1CAM, PI4K-A, MYO5, ANK1 and PURB, and combinations thereof.

In some embodiments, the method may further comprise a measure of an amount of FABP7, LMNB1, IGSF8, NCAM1, ANXA5, DCLK1, L1CAM, PI4K-A, MYO5, ANK1 and PURB.

An observed decrease of the amount of BDNF at step b) compared to step a) may be indicative of an efficacy of the therapeutic treatment against the condition.

An observed decrease of the amount of KIF1A at step b) compared to step a) may be indicative of an efficacy of the therapeutic treatment against the condition.

An observed increase of the amount of MECP2 at step b) compared to step a) may be indicative of an efficacy of the therapeutic treatment against the condition.

An observed increase of the amount of PLCB1 at step b) compared to step a) may be indicative of an efficacy of the therapeutic treatment against the condition.

An observed decrease of the amount of FABP7 at step b) compared to step a) may be indicative of an efficacy of the therapeutic treatment against the condition.

An observed decrease of the amount of LMNB1 at step b) compared to step a) may be indicative of an efficacy of the therapeutic treatment against the condition.

An observed increase of the amount of IGSF8 at step b) compared to step a) may be indicative of an efficacy of the therapeutic treatment against the condition.

An observed decrease of the amount of NCAM1 at step b) compared to step a) may be indicative of an efficacy of the therapeutic treatment against the condition.

An observed increase of the amount of ANXA5 at step b) compared to step a) may be indicative of an efficacy of the therapeutic treatment against the condition.

An observed decrease of the amount of DCLK1 at step b) compared to step a) may be indicative of an efficacy of the therapeutic treatment against the condition.

An observed decrease of the amount of L1CAM at step b) compared to step a) may be indicative of an efficacy of the therapeutic treatment against the condition.

An observed increase of the amount of PI4K-A at step b) compared to step a) may be indicative of an efficacy of the therapeutic treatment against the condition.

An observed decrease of the amount of MYO5 at step b) compared to step a) may be indicative of an efficacy of the therapeutic treatment against the condition.

An observed increase of the amount of ANK1 at step b) compared to step a) may be indicative of an efficacy of the therapeutic treatment against the condition.

An observed decrease of the amount of PURB at step b) compared to step a) may be indicative of an efficacy of the therapeutic treatment against the condition.

In some embodiments, various combined observations of the proteins indicated above may be carried out.

In some embodiments, an observed increase of the amounts of MeCP2 and PLCB1 and an observed decrease of the amount of KIF1A at step b) compared to step a) may be indicative of an efficacy of the therapeutic treatment against the condition.

In some embodiments, an observed increase of the amounts of MeCP2 and PLCB1 and an observed decrease of the amount of BDNF and KIF1A at step b) compared to step a) may be indicative of an efficacy of the therapeutic treatment against the condition.

In some embodiments, an observed decrease of the amounts of FABP7 and LMNB1 and an observed increase of the amount of IGSF8 at step b) compared to step a) may be indicative of an efficacy of the therapeutic treatment against the condition.

In some embodiments, an observed decrease of the amounts of NCAM1, DCLK1, L1CAM, MYO5 and PURB and an observed increase of the amounts of ANXA5, PI4K-A and ANK1 at step b) compared to step a) may be indicative of an efficacy of the therapeutic treatment against the condition.

In some embodiments, an observed increase of the amounts of MeCP2, PLCB1, IGSF8, ANXA5, PI4K-A and ANK1 and an observed decrease of the amounts of KIF1A, FABP7, LMNB1, NCAM1, DCLK1, L1CAM, MYO5 and PURB at step b) compared to step a) may be indicative of an efficacy of the therapeutic treatment against the condition.

In some embodiments, an observed increase of the amounts of MeCP2, PLCB1, IGSF8, ANXA5, PI4K-A and ANK1 and an observed decrease of the amounts of BDNF, KIF1A, FABP7, LMNB1, NCAM1, DCLK1, L1CAM, MYO5 and PURB at step b) compared to step a) may be indicative of an efficacy of the therapeutic treatment against the condition.

One objects of the disclosure relates to a method for selecting a candidate therapeutic agent for preventing and/or treating a cerebral creatine deficiency syndrome in an individual in need thereof, said method comprising the steps of:
a) measuring an amount of at least one protein selected from BDNF, KIF1A, MeCP2, PLCB1, and a combination thereof, in an isolated biological sample obtained from a biological model of a cerebral creatine deficiency syndrome before contacting said biological model with said candidate therapeutic agent,
b) measuring an amount of at least one protein selected from BDNF, KIF1A, MeCP2, PLCB1, and a combination thereof, in an isolated biological sample obtained from said biological model of step a) after contacting said biological model with said candidate therapeutic agent, the protein or combination of proteins of step a) and b) being the same,
c) comparing the amounts measured at step a) with the amounts measured at step b), and
d) selecting a candidate therapeutic agent for which a difference between the measured amounts obtained at step a) and at step b) is indicative of a therapeutic efficacy of said candidate therapeutic agent on said cerebral creatine deficiency syndrome.

In some embodiments, steps a) and b) comprise measuring an amount of the protein BDNF.

In some embodiments, steps a) and b) comprise measuring an amount of each protein KIF1A, MeCP2 and PLCB1. In some embodiments, steps a) and b) comprise further measuring an amount of the protein BDNF.

One objects of the disclosure relates to a method for selecting a candidate therapeutic agent susceptible to be used for preventing and/or treating a condition, the condition being a cerebral creatine deficiency syndrome in an individual in need thereof, said method comprising the steps of:
a) measuring an amount of each protein KIF1A, MeCP2, and PLCB1 in an isolated biological sample obtained from a biological model before contacting said biological model with a candidate therapeutic agent to be selected,
b) measuring an amount of each protein KIF1A, MeCP2, and PLCB1 in an isolated biological sample obtained from said biological model after contacting said biological model with said candidate therapeutic agent to be selected,
c) comparing the amounts measured at step a) with the amounts measured at step b), and
d) selecting a candidate therapeutic agent for which a difference between the measured amounts obtained at step a) and at step b) is indicative of a therapeutic efficacy of said candidate therapeutic agent on said cerebral creatine deficiency syndrome.

In some embodiments, the method may further comprise a measure of a further protein or a combination of proteins as above described.

In some embodiments, steps a) and b) comprise further measuring an amount of the protein BDNF.

In some embodiments, the method may further comprise a measure of an amount of at least one protein from the group FABP7, LMNB1, and IGSF8, and combinations thereof.

In some embodiments, the method may further comprise a measure of an amount of at least one protein from the group NCAM1, ANXA5, DCLK1, L1CAM, PI4K-A, MYO5, ANK1 and PURB, and combinations thereof.

In some embodiments, the method may further comprise a measure of an amount of at least one protein from the group FABP7, LMNB1, IGSF8, NCAM1, ANXA5, DCLK1, L1CAM, PI4K-A, MYO5, ANK1 and PURB, and combinations thereof.

In some embodiments, the method may further comprise a measure of an amount of FABP7, LMNB1, IGSF8, NCAM1, ANXA5, DCLK1, L1CAM, PI4K-A, MYO5, ANK1 and PURB.

An observed decrease of the amount of BDNF at step b) compared to step a) may be indicative of an efficacy of the candidate therapeutic agent against the condition.

An observed decrease of the amount of KIF1A at step b) compared to step a) may be indicative of an efficacy of the candidate therapeutic agent against the condition.

An observed increase of the amount of MECP2 at step b) compared to step a) may be indicative of an efficacy of the candidate therapeutic agent against the condition.

An observed increase of the amount of PLCB1 at step b) compared to step a) may be indicative of an efficacy of the candidate therapeutic agent against the condition.

An observed decrease of the amount of FABP7 at step b) compared to step a) may be indicative of an efficacy of the candidate therapeutic agent against the condition.

An observed decrease of the amount of LMNB1 at step b) compared to step a) may be indicative of an efficacy of the candidate therapeutic agent against the condition.

An observed increase of the amount of IGSF8 at step b) compared to step a) may be indicative of an efficacy of the candidate therapeutic agent against the condition.

An observed decrease of the amount of NCAM1 at step b) compared to step a) may be indicative of an efficacy of the candidate therapeutic agent against the condition.

An observed increase of the amount of ANXA5 at step b) compared to step a) may be indicative of an efficacy of the candidate therapeutic agent against the condition.

An observed decrease of the amount of DCLK1 at step b) compared to step a) may be indicative of an efficacy of the candidate therapeutic agent against the condition.

An observed decrease of the amount of L1CAM at step b) compared to step a) may be indicative of an efficacy of the candidate therapeutic agent against the condition.

An observed increase of the amount of PI4K-A at step b) compared to step a) may be indicative of an efficacy of the candidate therapeutic agent against the condition.

An observed decrease of the amount of MYO5 at step b) compared to step a) may be indicative of an efficacy of the candidate therapeutic agent against the condition.

An observed increase of the amount of ANK1 at step b) compared to step a) may be indicative of an efficacy of the candidate therapeutic agent against the condition.

An observed decrease of the amount of PURB at step b) compared to step a) may be indicative of an efficacy of the candidate therapeutic agent against the condition.

In some embodiments, various combined observations of the proteins indicated above may be carried out.

In some embodiments, an observed increase of the amounts of MeCP2 and PLCB1 and an observed decrease of the amount of KIF1A at step b) compared to step a) may be indicative of an efficacy of the candidate therapeutic agent against the condition.

In some embodiments, an observed increase of the amounts of MeCP2 and PLCB1 and an observed decrease of the amounts of BDNF and KIF1A at step b) compared to step a) may be indicative of an efficacy of the candidate therapeutic agent against the condition.

In some embodiments, an observed decrease of the amounts of FABP7 and LMNB1 and an observed increase of the amount of IGSF8 at step b) compared to step a) may be indicative of an efficacy of the candidate therapeutic agent against the condition.

In some embodiments, an observed decrease of the amounts of NCAM1, DCLK1, L1CAM, MYO5 and PURB and an observed increase of the amounts of ANXA5, PI4K-A and ANK1 at step b) compared to step a) may be indicative of an efficacy of the candidate therapeutic agent against the condition.

In some embodiments, an observed increase of the amounts of MeCP2, PLCB1, IGSF8, ANXA5, PI4K-A and ANK1 and an observed decrease of the amounts of KIF1A, FABP7, LMNB1, NCAM1, DCLK1, L1CAM, MYO5 and PURB at step b) compared to step a) may be indicative of an efficacy of the candidate therapeutic agent against the condition. A biological sample may be as disclosed herein.

For example, biological model may be a Slc6a8^{-/y} mouse, obtained as disclosed in the Examples section or in Raffaele, M. et al. Novel translational phenotypes and biomarkers for creatine transporter deficiency. Brain Commun., (2020)).

A biological model may also be cultured cells isolated from an individual suffering from a cerebral creatine deficiency syndrome. Such cells may be used in primary culture or after transformation of the cells in a cell line. For example, suitable cells can be fibroblast cells, for example isolated from the skin or from a muscle of an individual suffering from a cerebral creatine deficiency syndrome.

Also, a biological model may be a brain organoid obtained by dedifferentiation and reprogramming of fibroblast cells isolated from an individual suffering from a cerebral creatine deficiency syndrome as described above.

A method as disclosed herein may be for monitoring an evolution of a cerebral creatine deficiency syndrome in an individual in need thereof. Such method may comprise the steps of:
a) measuring an amount of at least one protein selected from BDNF, KIF1A, MeCP2, PLCB1, and a combination thereof, in an isolated biological sample obtained from said individual at a first time,
b) measuring an amount of at least one protein selected from BDNF, KIF1A, MeCP2, PLCB1, and a combination thereof, in an isolated biological sample obtained from said individual at a second time, subsequent to the first time, the protein or combination of proteins of step a) and b) being the same,
c) comparing the measured amounts obtained at step a) and at step b),
wherein a difference between the measured amounts may be indicative of an improvement or an aggravation of the cerebral creatine deficiency syndrome in said individual.

In some embodiments, steps a) and b) comprise measuring an amount of the protein BDNF.

In some embodiments, steps a) and b) comprise measuring an amount of each protein KIF1A, MeCP2 and PLCB1. In some embodiments, steps a) and b) comprise further measuring an amount of the protein BDNF.

A method as disclosed herein may be for monitoring an evolution of a condition, the condition being a cerebral creatine deficiency syndrome in an individual in need thereof. Such method may comprise the steps of:
a) measuring an amount of each protein KIF1A, MeCP2, and PLCB1 in an isolated biological sample obtained from said individual at a first time,
b) measuring an amount of each protein KIF1A, MeCP2, and PLCB1 in an isolated biological sample obtained from said individual at a second time, subsequent to the first time,
c) comparing the measured amounts obtained at step a) and at step b),
wherein an observed difference between the measured amounts between step a) and step b) may be indicative of an improvement or an aggravation of the cerebral creatine deficiency syndrome in said individual.

In some embodiments, the method may further comprise a measure of a further protein or a combination of proteins as above described.

In some embodiments, steps a) and b) comprise further measuring an amount of the protein BDNF.

In some embodiments, the method may further comprise a measure of an amount of at least one protein from the group FABP7, LMNB1, and IGSF8, and combinations thereof.

In some embodiments, the method may further comprise a measure of an amount of at least one protein from the group NCAM1, ANXA5, DCLK1, L1CAM, PI4K-A, MYO5, ANK1 and PURB, and combinations thereof.

In some embodiments, the method may further comprise a measure of an amount of at least one protein from the group FABP7, LMNB1, IGSF8, NCAM1, ANXA5, DCLK1, L1CAM, PI4K-A, MYO5, ANK1 and PURB, and combinations thereof.

In some embodiments, the method may further comprise a measure of an amount of FABP7, LMNB1, IGSF8, NCAM1, ANXA5, DCLK1, L1CAM, PI4K-A, MYO5, ANK1 and PURB.

An observed increase of the amount of BDNF may be indicative of an aggravation of the condition.

An observed decrease of the amount of BDNF may be indicative of an improvement of the condition.

An observed increase of the amount of KIF1A may be indicative of an aggravation of the condition.

An observed decrease of the amount of KIF1A may be indicative of an improvement of the condition.

An observed decrease of the amount of MECP2 may be indicative of an aggravation of the condition.

An observed increase of the amount of MECP2 may be indicative of an improvement of the condition.

An observed decrease of the amount of PLCB1 may be indicative of an aggravation of the condition.

An observed increase of the amount of PLCB1 may be indicative of an improvement of the condition.

An observed increase of the amount of FABP7 may be indicative of an aggravation of the condition.

An observed decrease of the amount of FABP7 may be indicative of an improvement of the condition.

An observed increase of the amount of LMNB1 may be indicative of an aggravation of the condition.

An observed decrease of the amount of LMNB1 may be indicative of an improvement of the condition.

An observed decrease of the amount of IGSF8 may be indicative of an aggravation of the condition.

An observed increase of the amount of IGSF8 may be indicative of an improvement of the condition.

An observed increase of the amount of NCAM1 may be indicative of an aggravation of the condition.

An observed decrease of the amount of NCAM1 may be indicative of an improvement of the condition.

An observed decrease of the amount of ANXA5 may be indicative of an aggravation of the condition.

An observed increase of the amount of ANXA5 may be indicative of an improvement of the condition.

An observed increase of the amount of DCLK1 may be indicative of an aggravation of the condition.

An observed decrease of the amount of DCLK1 may be indicative of an improvement of the condition.

An observed increase of the amount of L1CAM may be indicative of an aggravation of the condition.

An observed decrease of the amount of L1CAM may be indicative of an improvement of the condition.

An observed decrease of the amount of PI4K-A may be indicative of an aggravation of the condition.

An observed increase of the amount of PI4K-A may be indicative of an improvement of the condition.

An observed increase of the amount of MYO5 may be indicative of an aggravation of the condition.

An observed decrease of the amount of MYO5 may be indicative of an improvement of the condition.

An observed decrease of the amount of ANK1 may be indicative of an aggravation of the condition.

An observed increase of the amount of ANK1 may be indicative of an improvement of the condition.

An observed increase of the amount of PURB may be indicative of an aggravation of the condition.

An observed decrease of the amount of PURB may be indicative of an improvement of the condition.

In some embodiments, various combined observations of the proteins indicated above may be carried out.

In some embodiments, an observed decrease of the amounts of MeCP2 and PLCB1 and an observed increase of the amount of KIF1A may be indicative of an aggravation of the condition.

In some embodiments, an observed increase of the amounts of MeCP2 and PLCB1 and an observed decrease of the amount of KIF1A may be indicative of an improvement of the condition.

In some embodiments, an observed decrease of the amounts of MeCP2 and PLCB1 and an observed increase of the amounts of BDNF and KIF1A may be indicative of an aggravation of the condition.

In some embodiments, an observed increase of the amounts of MeCP2 and PLCB1 and an observed decrease of the amounts of BDNF and of KIF1A may be indicative of an improvement of the condition.

In some embodiments, an observed increase of the amounts of FABP7 and LMNB1 and an observed decrease of the amount of IGSF8 may be indicative of an aggravation of the condition.

In some embodiments, an observed decrease of the amounts of FABP7 and LMNB1 and an observed increase of the amount of IGSF8 may be indicative of an improvement of the condition.

In some embodiments, an observed increase of the amounts of NCAM1, DCLK1, L1CAM, MYO5 and PURB and an observed decrease of the amounts of ANXA5, PI4K-A and ANK1 may be indicative of an aggravation of the condition.

In some embodiments, an observed decrease of the amounts of NCAM1, DCLK1, L1CAM, MYO5 and PURB and an observed increase of the amounts of ANXA5, PI4K-A and ANK1 may be indicative of an improvement of the condition.

In some embodiments, an observed decrease of the amounts of MeCP2, PLCB1, IGSF8, ANXA5, PI4K-A and ANK1 and an observed increase of the amounts of KIF1A, FABP7, LMNB1, NCAM1, DCLK1, L1CAM, MYO5 and PURB may be indicative of an aggravation of the condition.

In some embodiments, an observed increase of the amounts of MeCP2, PLCB1, IGSF8, ANXA5, PI4K-A and ANK1 and an observed decrease of the amounts of KIF1A, FABP7, LMNB1, NCAM1, DCLK1, L1CAM, MYO5 and PURB may be indicative of an improvement of the condition.

In some embodiments, an observed decrease of the amounts of MeCP2, PLCB1, IGSF8, ANXA5, PI4K-A and ANK1 and an observed increase of the amounts of BDNF, KIF1A, FABP7, LMNB1, NCAM1, DCLK1, L1CAM, MYO5 and PURB may be indicative of an aggravation of the condition.

In some embodiments, an observed increase of the amounts of MeCP2, PLCB1, IGSF8, ANXA5, PI4K-A and ANK1 and an observed decrease of the amounts of BDNF, KIF1A, FABP7, LMNB1, NCAM1, DCLK1, L1CAM, MYO5 and PURB may be indicative of an improvement of the condition.

A method for monitoring an evolution of a cerebral creatine deficiency syndrome may comprise in addition to the 1^{st} and 2^{nd} measures further subsequent measures taken at subsequent times. For example, a method may comprise a 3^{rd}, a 4^{th}, a 5^{th}, a 6^{th}, a 7^{th}, an 8^{th}, a 9^{th}, a 10^{th} or more measure.

In a method disclosed herein the measures may be repeated at regular or irregular intervals. For example, the measures may be carried every day, once a week every week, or every 2, 3, or 4 weeks, once a month every month, or every 2, 3, 4, 5, 6, 7, 8, 9, 10, 11 months, or once year every year, or every 2, 3, or 4 years. The measures may be carried several times a week, a month or a year, for example, twice, or three, four, five or six time a week, or more in month or in a year. The measures may be carried out over a period of time ranging from one week to one year, or more.

A cerebral creatine deficiency syndrome concerned by the disclosed methods may be a creatine transporter (CRTR) deficiency.

According to another of its objects, the present disclosure relates to a method of diagnosing and treating an individual susceptible to suffer from a cerebral creatine deficiency syndrome, the method comprising the steps of:
a) measuring an amount of at least one protein selected from BDNF, KIF1A, MeCP2, PLCB1, and a combination thereof, in an isolated biological sample obtained from said individual,
b) comparing each amount measured at step a) with a predetermined reference value of said proteins,
wherein a difference between the measured amounts and the predetermined reference values may provide a diagnosis of a cerebral creatine deficiency syndrome in said individual, and
c) administering to said individual diagnosed with a cerebral creatine deficiency syndrome a therapeutic treatment proposed for preventing and/or treating a cerebral creatine deficiency syndrome.

In some embodiments, step a) comprises measuring an amount of the protein BDNF.

In some embodiments, step a) comprises measuring an amount of each protein KIF1A, MeCP2 and PLCB1. In some embodiments, step a) comprises further measuring an amount of the protein BDNF.

According to another of its objects, the present disclosure relates to a method of diagnosing and treating an individual susceptible to suffer from a cerebral creatine deficiency syndrome, the method comprising the steps of:
a) measuring an amount of each protein KIF1A, MeCP2, and PLCB1 in an isolated biological sample obtained from said individual,
b) comparing each amount measured at step a) with a predetermined reference value of said proteins,
wherein a difference between the measured amounts and the predetermined reference values may provide a diagnosis of a cerebral creatine deficiency syndrome in said individual, and
c) administering to said individual diagnosed with a cerebral creatine deficiency syndrome a therapeutic treatment proposed for preventing and/or treating a cerebral creatine deficiency syndrome.

In some embodiments, step a) comprises further measuring an amount of the protein BDNF.

The diagnosing part of the method disclosed herein may be carried out as above described.

Suitable treatment for an individual diagnosed with a cerebral creatine deficiency syndrome, such a CTD, may include occupational, speech, and physical therapies to treat developmental disabilities and behavioral therapy to address behavior problems.

Suitable treatments with therapeutic agents which may be implemented include creatine monohydrate, L-arginine, glycine, dodecyl creatine ester, creatine analog such as cyclocreatine, and combinations thereof.

In some embodiments, a therapeutic agent may be dodecyl creatine ester.

In some embodiments, a therapeutic agent may be dodecyl creatine ester incorporated into lipid nanocapsules.

In some embodiments, the present disclosure relates to a method for selecting a protein or a set of proteins as a biomarker of a cerebral creatine deficiency syndrome.

A method of the disclosure may comprise the steps of:
a) measuring an amount for each protein of a set of a plurality of proteins in a biological sample obtained from at least one individual suffering from a cerebral creatine deficiency syndrome,
b) measuring an amount for each protein of a set of a plurality of proteins in a biological sample obtained from at least one healthy individual,
c) normalizing data of the proteins, for example using the Variance Stabilizing Normalization, and filtering,
d) identifying and selecting proteins differentially expressed by comparing the normalized data of step a) with the normalized data of step b) with a statistical model,
e) subjecting the proteins selected at step d) to a pathway analysis using gene set enrichment and selecting relevant pathways based on a p<0.05 cut-off,
f) statistical modelling of the differentially expressed proteins, for example with a multivariate statistical model using one-way ANOVA with Bonferroni post-hoc analysis comparison,
g) selecting the proteins significantly altered in the individual suffering from a cerebral creatine deficiency syndrome.

The method may further comprise a step of correlating the selected proteins at step g) with results of at least one cognition test. This step may be carried out with a stepwise regression model.

The method may comprise an additional set of measures of amounts of proteins obtained from individual suffering from a cerebral creatine deficiency syndrome receiving a treatment against a cerebral creatine deficiency syndrome.

In some embodiments, the individuals may be wild-type mice and mice modelling a cerebral creatine deficiency syndrome.

It shall be understood that the features mentioned above and those to be mentioned in the following cannot only be used in the combinations indicated in each case but also in other combinations or in isolated positions without departing from the scope of the present disclosure.

The disclosure is now explained in more details by means of embodiments which result in further characteristics, features and advantages. The embodiments are purely illustrative and do not restrict the scope of the disclosure. Features which are described in connection with a specific embodiment are isolated features of the disclosure in its general form and are not only features in the specific technical context.

### EXAMPLES

### Example 1: Materials and Methods

### Generation of CrT (Creatine Transporter) KO (Knocked-Out) Mice

All in vivo experiments were conducted in compliance with the European Communities Council Directive of 22 Sep. 2010 and were approved by the Italian Ministry of Health (authorization number 259/2016-PR). CrT^{-/y} and CrT^{+/y} male mice were generated on a C57BL/6J background as described previously (Baroncelli L, et al. A novel mouse model of creatine transporter deficiency. F1000Res 3, 228 (2014)). Mice were kept at 22° C. under a 12-12 h light-dark cycle and received food and water ad libitum. The Slc6a8 mutation was confirmed by PCR as previously described (Baroncelli L, et al. A mouse model for creatine transporter deficiency reveals early onset cognitive impairment and neuropathology associated with brain aging. Human Molecular Genetics 25, 4186-4200 (2016)). Genomic DNA was isolated from mouse-tail tissue using the DNeasy® Blood & Tissue kit from Qiagen according to the manufacturer protocol. The following primers were used for PCR amplification: F: AGGTTTCCTCAGGTTATAGAGA (SEQ ID NO: 1); R: CCCTAGGT GTATCTAACATCT (SEQ ID NO: 2); R1: TCGTGGTATCGTTATGCGCC (SEQ ID NO: 3). Amplicon sizes were as follows: Crt^{+/y} allele=462 bp; mutant allele=371 bp.

### Drugs

We used dodecyl creatine ester (DCE) as drug. DCE was synthesized as described (Trotier-Faurion, A. et al. Synthesis and Biological Evaluation of New Creatine Fatty Esters Revealed Dodecyl Creatine Ester as a Promising Drug Candidate for the Treatment of the Creatine Transporter Deficiency. Journal of Medicinal Chemistry 56, 5173-5181, (2013).). Ten (10) mg of DCE was added to 0.375 g of room temperature Maisine® CC (Gattefossé) and then 125 mg of docosahexaenoic acid (DHA) (Sigma-Aldrich) was incorporated. The mixtures were vortexed for 5 min and shaken at 1000 g in a thermomixer at 30° C. for 48 h. Then, the sample was centrifuged at 20,000 g for 10 min at room temperature and the resulting supernatant was filtered through a 0.22 μm filter, placed in another tube and stored at ±4° C. prior to use.

### Behavioral Testing

Behavioral testing started 14 after the start of treatment (Baroncelli L, et al. A mouse model for creatine transporter deficiency reveals early onset cognitive impairment and neuropathology associated with brain aging. Human Molecular Genetics 25, 4186-4200 (2016)). Treatment continued during behavioral testing, which took two weeks, for a total of 30 days of treatment. The testing order for behavioral assessment performed in the same mice consisted of: object recognition test (ORT) 24 h (3 days), Y maze (1 day), Morris water maze (MWM) with hidden platform (7 days).

### Object Recognition Test

Mice were tested in a poly-vinyl chloride square arena (60×60×30 cm) with black walls and a white floor as previously described (Baroncelli L, et al. A mouse model for creatine transporter deficiency reveals early onset cognitive impairment and neuropathology associated with brain aging. Human Molecular Genetics 25, 4186-4200 (2016)). The day before testing, mice were familiarized with the empty arena for 10 min. The object recognition test (ORT), which is based on the spontaneous tendency of rodents to spend more time exploring a novel object than a familiar one, measures short- and long-term memory and consists of a sample and a testing phase. During the sample phase, mice were placed in the arena with two identical objects placed in diagonally opposite corners of the arena (about 6 cm from the walls) for 10 min. The test phase was 24 hours after the sample phase. Mice were returned to the arena with an identical copy of one of the familiar objects and a new object placed in the same position. The mice explored the objects for 5 min. A discrimination index was computed as: DI=(T new−T old)/(T new+T old), where T new is the time spent exploring the new object, and T old is the time spent exploring the old one (Baroncelli L, et al. A mouse model for creatine transporter deficiency reveals early onset cognitive impairment and neuropathology associated with brain aging. Human Molecular Genetics 25, 4186-4200 (2016)).

### Y Maze Spontaneous Alternation

Spontaneous alternation was measured using a Y-shaped maze with three symmetrical grey solid plastic arms at a 120-degree angle (26 cm length, 10 cm width, and 15 cm height) as described (Baroncelli L, et al. A mouse model for creatine transporter deficiency reveals early onset cognitive impairment and neuropathology associated with brain aging. Human Molecular Genetics 25, 4186-4200 (2016); Begenisic T, et al. Fluoxetine in adulthood normalizes GABA release and rescues hippocampal synaptic plasticity and spatial memory in a mouse model of Down syndrome. Neurobiol Dis 63, 12-19 (2014)). Mice were placed in the center of the maze and movement was recorded for 8 min. The number of arm entries (all four limbs within the arm) and the number of triads (three arm entries) were video-recorded in order to calculate the alternation percentage defined as the number of triads divided by the number of possible alternations (total arm entries minus 2) and then multiplying by 100.

### Morris Water Maze (MWM)

Mice were trained for 4 trials per day and for a total of 7 days in a circular water tank (diameter, 120 cm; height, 40 cm), filled with water (23° C.) rendered opaque by the addition of a non-toxic white paint to a depth of 25 cm. Four positions were arbitrarily designated North (N), South(S), East (E), and West (W), providing 4 start positions and defining the partition of the tank into 4 quadrants. A square escape platform (11×11 cm) was submerged 0.5 cm below the water and placed at the midpoint of one of the 4 quadrants. Mice were allowed up to 60 s to reach the escape platform, and their swimming paths were automatically recorded by the Noldus Ethovision system. On the last trial of the last training day, mice received a probe trial, during which the escape platform was removed from the tank and the swimming paths were recorded over 60 s while mice searched for the missing platform

### Protein Proteolysis and Mass Spectrometry Analysis

Total protein (15 μg) was extracted from different brain regions and mixed with lithium dodecyl sulfate lysis buffer (Invitrogen) and incubated at 99° C. for 5 min, and then separated by a short electrophoresis migration (5 min) at 200 V on NuPAGE 4-12% Bis-Tris gel with MES/SDS 1× (Invitrogen) as running buffer. Gels were stained with SimplyBlue SafeStain (Thermo) for 5 min followed by an overnight wash in water with gentle agitation. The polyacrylamide band containing the whole proteome from each sample was excised and treated as recommended (Hartmann E M, Armengaud J. N-terminomics and proteogenomics, getting off to a good start. Proteomics 14, 2637-2646 (2014)). Proteins were in-gel proteolyzed with trypsin gold (Promega) in the presence of 0.01% of Protease Max surfactant (Promega) at 50° C. for 60 min. From the resulting peptide fraction (50 μL), a volume of 1 μL corresponding to approximately 300 ng of peptides was analyzed by liquid chromatography-tandem mass spectrometry (LC-MS/MS) using an Ultimate 3000 nano-LC system coupled to a Q-Exactive HF mass spectrometer (Thermo Scientific) operated as described previously (Hartmann E M, Armengaud J. N-terminomics and proteogenomics, getting off to a good start. Proteomics 14, 2637-2646 (2014)). Peptides were loaded on a reverse-phase PepMap 100 C18 μ-precolumn (5 μm, 100 Å, 300 μm i.d.×5 mm, Thermo Fisher), and then resolved on a nanoscale PepMap 100 C18 nanoLC column (3 μm, 100 Å, 75 μm i.d.×50 cm, Thermo Fisher) at a flow rate of 0.2 μL·min⁻¹ using a 90-min gradient (4% B for 0 to 3 min, 4-25% B from 3 to 78 min and 25-40% B from 78 to 93 min) with mobile phase A corresponding to 0.1% HCOOH/100% H₂O and mobile phase B corresponding to 0.1% HCOOH/80% CH₃CN/20% H₂O). The mass spectrometer was operated in Top20 mode, with a scan range of MS acquisition from 350 to 1800 m/z and selection and fragmentation using a 10 s dynamic exclusion time for the 20 most abundant precursor ions. Only ion precursors with a 2+ or 3+ charge were selected for High-energy collisional dissociation (HCD) fragmentation performed at a normalized collision energy of 27 eV.

### MS/MS Spectra Interpretation and Differential Proteomics.

MS/MS spectra were assigned using the Mascot Daemon software version 2.6.1 (Matrix Science) and the Mus musculus SwissProt database comprising 17,096 protein sequences. Peptide tolerance, MS/MS fragment tolerance, and the maximum of missed cleavages were set at 5 ppm, 0.02 D and 2, respectively. Carbamidomethylation of cysteine was considered as fixed modification. Oxidation of methionine was taken into account as variable modification. Peptides identified at a p-value≤0.05 in homology threshold mode and proteins identified with at least two distinct peptides were selected (false discovery rate below 1%).

### Bioinformatics Analysis of Proteomic Data

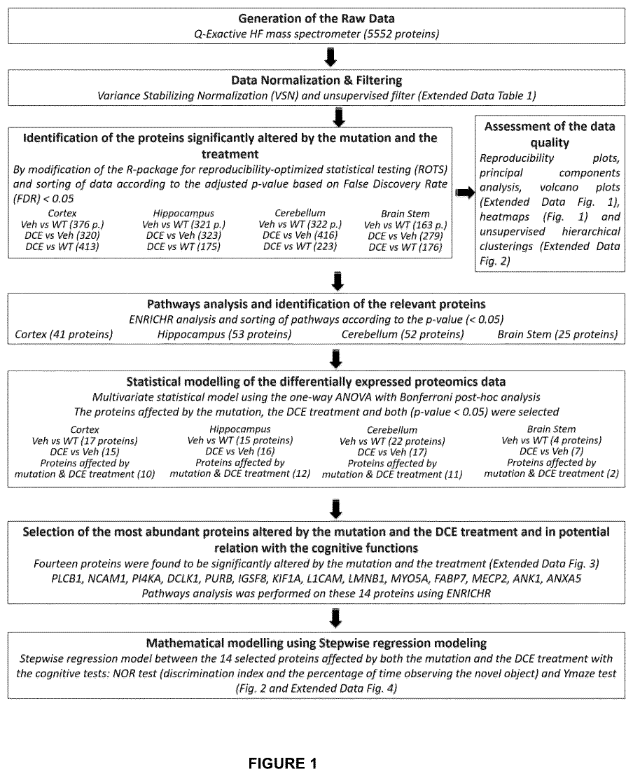

The general workflow is shown in FIG. 1. In-house script was constructed, using R programming language, to identify the differentially expressed proteins between the muscle and four different regions of the brain: cortex, cerebellum, hippocampus and brainstem. Initially, the proteomics data were normalized using the Variance Stabilizing Normalization (Motakis E S, Nason G P, Fryzlewicz P, Rutter G A. Variance stabilization and normalization for one-color microarray data using a data-driven multiscale approach. Bioinformatics 22, 2547-2553 (2006)). Proteins with at least 10 MS/MS spectra across all conditions were retained. Unsupervised variation filter was then applied to the proteomics data (Hamoudi R A, et al. Differential expression of NF-kappaB target genes in MALT lymphoma with and without chromosome translocation: insights into molecular mechanism. Leukemia 24, 1487-1497 (2010)) where any 8 samples with MS/MS detected protein were included. The differential abundance analysis for the proteins between the different regions was carried out using modification to the R package for Reproducibility Optimized Statistical Testing (Suomi T, Seyednasrollah F, Jaakkola M K, Faux T, Elo L L. ROTS: An R package for reproducibility-optimized statistical testing. Plos Computational Biology 13, (2017)). The data were sorted according to the adjusted p-value based on false discovery rate <0.05. Reproducibility plots and principal component analysis were used to assess the quality of the separation of the data between the various groups being compared. The identified differentially expressed proteins were visualized using volcano plots and heatmaps. The heatmaps were generated using unsupervised hierarchical clustering carried out with Ward linkage and Euclidean distance measure to assess the degree of proteomic profile separation between the three groups across the four brain regions.

### Pathways Analysis

Many proteins play a part in the same pathway. Therefore, in order to reduce the set of differentially expressed proteins and identify potential functions of the different expressed proteins, the proteins significantly altered by the mutation compared to the WT (wild-type), and by the treatment compared to vehicle (Veh) were selected for a subsequent pathway analysis. The proteins that were altered by the vehicle and the treatment compared to the WT were also selected. Pathway analysis using gene set enrichment was carried out using Enrichr (Chen E Y, et al. Enrichr: interactive and collaborative HTML5 gene list enrichment analysis tool. BMC Bioinformatics 14, 128 (2013); Kuleshov M V, et al. Enrichr: a comprehensive gene set enrichment analysis web server 2016 update. Nucleic Acids Res 44, W90-97 (2016)) focusing on the following sets: BioCarta_2016, Elsevier_Pathway_Collection, GO_Biological_Process_2018, GO_Molecular_Function_2018, KEGG_2019_Human, KEGG_2019_Mouse, MSigDB_Hallmark_2020, WikiPathways_2019_Mouse, WikiPathways_2019_Human, ClinVar_2019, DisGeNET, Jensen_DISEASES, OMIM_Disease. Relevant pathways are selected based on a p<0.05 cut-off.

### Statistical Modelling of the Differentially Expressed Proteomics Data

In order to identify the patterns of differentially expressed proteins across the different regions, the normalized data of the proteins identified from the pathway analysis were used to construct a multivariate statistical model using one-way ANOVA with Bonferroni post-hoc analysis comparison across the following groups: cortex, cerebellum, brainstem, hippocampus and muscle. The most abundant proteins altered by the mutation and the DCE treatment were selected. In order to correlate the proteins involved in cognition, a stepwise regression model was constructed to correlate these differentially expressed proteins with each of the following cognitive tests: NOR test (discrimination index and the percentage of time spent observing the novel object) and the Y-maze test. The results were further validated using Pearson correlation of the proteins identified across the different group.

### Western Blotting

Western blotting was used to detect the abundance of PLCB1 and the two inhibitors of NFkB, IKBα and IKBβ, in the dissected brain tissue. Briefly, brain tissue was homogenized in freshly prepared lysis buffer containing 20 mM Trizma-Base, 150 mM NaCl, pH 7.4 (Sigma-Aldrich, Saint-Quentin Fallavier, France) and supplemented with 1% Triton X-100, 4% of complete Protease Inhibitor Cocktail and 20% of a mix of anti-phosphatase inhibitors using a Precellys Evolution tissue homogenizer. The samples were then centrifuged first at 2500×g (15 min) and then at 10 000×g (20 min) on homogenates to produce lysate for electrophoresis. Proteins (10 to 20 μg) and protein standard in Laemmli buffer were loaded on 4%-15% CRITERION TGX STAIN-FREE protein gel in running buffer TGS 1×(all from Bio-Rad, Marnes-la-Coquette, France) and transferred to a 0.2 μm PVDF membrane with the Trans-Blot Turbo RTA Midi Transfer Kit (Bio-Rad, Marnes-la-Coquette, France). Membranes were blocked for 30 min in 5% low-fat milk in TBS-Tween 20 0.1% at room temperature. Blots were probed with specific primary antibodies overnight at 4° C. and detected by horseradish peroxidase secondary antibodies diluted 1:5 000 or 1:50 000 in 5% low-fat milk in TBS-Tween 20 0.1% at room temperature. For protein detection, membranes were exposed to the ECL prime Western blotting system (Amersham, UK) or Clarity western ECL substrate in a chemidoc touch imaging system for a measurable exposure time (Bio-Rad, Marnes-la-Coquette, France) and quantified with Image Lab Software (Bio-Rad, Marnes-la-Coquette, France). The following antibodies and dilutions were used: anti-PLCB1 (1/1000, Abcam, ab182359), anti-IKBα (1/500, Cell Signaling Technology, 4812S), anti-IKBβ (1/500, Cell Signaling Technology, 15519S), anti-PSD95 (1/2000, Merck, MABN68), and anti-tubulin (1/2000, Sigma-Aldrich, T6199), anti-KIF1A (1/1000, Abcam, ab180153).

### Co-Immunoprecipitation

200 μg of protein extracts were adjusted to a final volume of 600 μl with binding buffer (20 mM Tris-HCl, pH 7.5, 150 mM NaCl, 10% glycerol, 1 mM EDTA, 0.1% BSA, 1× protease inhibitor) before addition of 17.6 μl of the corresponding anti-protein antibody and 100 Units of Benzonase nuclease (Novagen 70746-3). The mix was incubated overnight at 4° C. on a rotating wheel. 43 μl of Dynabeads protein G (Invitrogen 10003D) were washed 3 times in PBS+0.05% Tween and once with binding buffer. Beads were then added to the IP reaction and incubated for 1 hour at room temperature with rotation. After incubation, the Ip reaction was washed twice with Benzonase buffer (20 mM Tris-HCl, pH 8.0, 20 mM NaCl, 10% glycerol, 2 mM MgCl₂, 0.1% BSA, 1× Protease inhibitor (Roche)) and incubated in Benzonase buffer supplemented with 100 U of Benzonase nuclease for 30 min at 37° C. before being washed three times with washing buffer (20 mM Tris-HCl, pH 7.5, 150 mM NaCl, 10% glycerol, 1 mM EDTA, 0.05% Tween, 1× protease inhibitor). Immunoprecipitated proteins were eluted directly in 25 μl of 1.5× Laemmli buffer supplemented with 200 mM DTT and 1 mM beta-mercaptoethanol with heat at 95° C. for 10 min before magnetic separation of beads and mass spectrometry analysis. Mass spectrometry analysis was done in similar conditions as for the brain extracts, except that the nano-UPLC gradient was reduced to 60-min.

### Protein Expression, Purification and Characterization

### Plasmid Generation.

DNA cassettes containing mouse's MeCP2 isoform 1 or PLCB1 sequence were generated, in-frame with nucleotides coding for C-terminal Histidine (HHHHHH) (SEQ ID NO: 6) or Myc (EQKLISEEDL) (SEQ ID NO: 7) peptides tags, respectively. The two inserts were designed to harbor an upstream 5′ adaptor sequence 5′ NTsc (5′-GTAGCAACAGCTACAGGCGCGCACTCC-insert-3′) (SEQ ID NO: 4) and a downstream CTsc (5′-insert-TAATGAGGGATCCCCCGACCTCGAC CTCTGGC-3′) 3′ adaptor sequence (SEQ ID NO: 5). The adaptor DNA sequences introduced a N-term BssHII and a C-term BamHI restriction sites for directional subcloning. The full-length DNA cassettes were generated by DNA synthesis (Twist Bioscience). The synthesized DNA constructs were subcloned into the pTT5 mammalian expression vector (DNA2.0, Inc., USA) pre-digested at BssHII and BamHI sites, using the In-Fusion Cloning kit (Takara Bio, Inc) according to manufacturer's instructions.

After transformation in Stellar competent cells (Takara Bio, Inc), low endotoxin plasmids (pMBE-MeCP2-6×His tag or pMBE-PLCB1-Myc) resulting from Plasmid Plus Maxi kit (Qiagen) were checked by sequence analyses.

### Protein Expression and Purification.

Recombinant MeCP2 and PLCB1 proteins were produced by transient expression in human cell lines HEK293 obtained from American Type Culture Collection (ATCC) and maintained in DMEM supplemented with 10% FBS, penicillin-streptomycin and L-glutamine (Gibco) in a 37C, 5% CO2 humidified incubator. Cells were transfected with plasmids coding for MeCP2-His or PLCB1-Myc c-terminal tags cassettes using Lipofectamine 3000 (Thermo Fisher Scientific). HEK293 transfection enhancer reagent was added to the cells 12-24 hours after transfection, according to manufacturer's instruction. The cultures were harvested 72 h after activation.

### Expression Cultures Fractionations.

Cells' cultures supernatants were fractioned by centrifugation. Cells' pellets were washed twice in ice-cold PBS and resuspended in ice-cold Buffer A: 10 mmol/L HEPES pH 7.9, 10 mmol/L KCl, 0.1 mol/L EDTA, and 0.5 mol/L EGTA. The pellets were lysed by adding NP-40 to 0.8% to buffer A with 10 seconds of vortexing, then centrifuged and the supernatant containing the cytoplasmic extract was collected. The nuclear pellet was resuspended in Buffer C: N-Buffer (20 mmol/L Tris pH 7.5, 100 mmol/L KCl, 2 mmol/L MgCl2, 1 mmol/L CaCl2, 0.3 mol/L sucrose, 0.1% Triton X-100, and 3 U/mL micrococcal nuclease), dounced, sonicated (10 minutes; 70% amplitude) in a Q700 cup horn and then incubated at RT for 15 minutes for DNase I digestion. Chromatin extracts reactions were stopped by the addition of EGTA (5 mmol/L) clarified by centrifugation and passed through 0.2 μm cellulose syringe filters device (Thomas Scientific, USA). The His-tagged MeCP2-His proteins were purified on a HisPur™ Cobalt 3 ml column (Thermo Fisher Scientific). The bound samples were washed with: 50 mM sodium phosphate, 300 mM sodium chloride, 10 mM imidazole; pH 7.4 and eluted with 50 mM sodium phosphate, 300 mM sodium chloride, 150 mM imidazole; pH 7.4. Elution fractions were analyzed by sodium dodecyl sulfate polyacrylamide gel electrophoresis (SDS-PAGE) and Coomassie-staining and appropriate fractions were pooled. The PLCB1-myc tagged proteins were enriched by the μMACS c-myc isolation kit, in a non-denaturing elution of the column bound antigens using pH shift with triethylamine at pH 11.8 (Miltenyi Biotec). Repeated μMACS eluted fraction were pooled and the concentration measured by Bradford assay. Finally, the proteins were concentrated, and buffer exchanged to 1×PBS, pH 7.4 with Amicon® Ultra-15 (Merck-Millipore) and Vivaspins® 20 (Cytiva) 10 kDa-pore size ultrafiltration devices. Protein concentration was measured by Pierce™ BCA Protein Assay kit (Thermo Fisher Scientific) and Nanodrop reads at 280 nm (Thermo Fisher Scientific). Finally, 4-10 μg of protein pooled fractions were analyzed by SDS-PAGE and Coomassie-stained. The samples were stored at −20° C. until used.

### Antigen Specific Enzyme-Linked Immunosorbent Assay (ELISA).

One hundred (100) microliters of MeCP2-His (2 μg/ml) or PLCB1-myc (2 μg/ml) proteins was coated into MAXISORP ELISA plate (Life Technology) and incubated for 15 hours at 4° C. Plates were washed three times with 300 μl/well of 1×PBS, 0.05% Tween20 (PBST) and blocked with 300 μL/well PBST supplemented with 5.0% Non-fat Dry Milk (blocking buffer) for 2 hours at room temperature. The plates were then washed three times with 300 μl PBST. Secondary antibody (horseradish peroxidase) (HRP) conjugated Rabbit anti-MeCP2 Polyclonal (LS-C445360) Antibody (LSBio) or Mouse anti-PLCB1 Monoclonal (LS-C548760) Antibody (LSBio) and assay controls HRP-labeled anti-His tag MAB050H Antibody (R&D system) or Myc.A7 Tag (HRP) Monoclonal antibody (Thermo Fisher Scientific) diluted in blocking buffer, were serial-diluted (1:3) starting at 2 μg/ml in blocking buffer. One hundred (100) microliters of sample were then added to each well and plates incubated for 2 hours at room temperature, 100 rpm rocking. The plates were washed three times with 300 μl/well of PBST. Plates were developed using 100 μL/well of 3,3′,5,5′-tetramethylbenzidine (TMB, Thermo Fischer Scientific) substrate for 15 min at room temperature. The reaction was stopped by adding 100 μL/well of ELISA Stop Solution (Thermo Fisher) and the optical densities were read at 450 nm.

### Protein-Protein Interaction ELISA.

MAXISORP ELISA plate (Life Technology) was coated with 2 μg/ml of the fusion protein MeCP2-6×His, diluted in ELISA coating buffer (Candor Bioscience) for 15 hours at 4° C. The plate was washed three times with 300 μL/well of PBST, then blocked with 300 μL/well of PBST, 5.0% Non-fat Dry Milk (blocking buffer) for 2 hours at room temperature. Two set of serial-dilutions (1:3; starting at 2 μg/ml) of the fusion protein PLCB1-Myc or Myc.A7 Tag (HRP) Monoclonal antibody (Thermo Fisher) diluted in blocking buffer were generated. after washing the plate three times with 300 μL of PBST, 100 μL of diluted sample were added to each well. The plates were incubated for 2 hours at room temperature. Following three times plates washes with PBST, wells incubated with PLCB1-Myc proteins were incubated with 100 μL of HRP-labeled Myc.A7 Tag (HRP) Monoclonal antibody (1:2000 dilution, Thermo Fisher) in blocking buffer for 1 hour at room temperature. Plates were developed using 100 μL/well of TMB substrate for 15 min at room temperature. The reaction was stopped by adding 100 μL/well of ELISA Stop Solution (Thermo Fisher) and the optical densities were read at 450 nm.

### Example 2: Results

The data presented here highlights several molecular targets that were disrupted in the brain of Slc6a8^{-/y} mice (Raffaele, M. et al. Novel translational phenotypes and biomarkers for creatine transporter deficiency (CTD). Brain Commun., (2020)) and modulated by dodecyl creatine ester (DCE) treatment. Furthermore, these protein changes are correlated to cognitive performance.

DCE was intranasally administered (Ullio-Gamboa G, et al. Dodecyl creatine ester-loaded nanoemulsion as a promising therapy for creatine transporter deficiency. Nanomedicine 14, 1579-1593 (2019)) to Slc6a8^{-/y} for 30 days, once a day at 4 mg/g of body weight, whilst the control groups were wild-type (WT) and vehicle-treated (Veh) Slc6a8^{-/y} mice, at N=8/group. Cognitive performance was evaluated using the object recognition test, the Y-maze, and the Morris water maze (MWM). Then, proteomics was performed on four different brain regions: cortex, hippocampus, cerebellum and brainstem; and the key protein abundances were correlated with behavioral performance, identifying several mechanistic pathways.

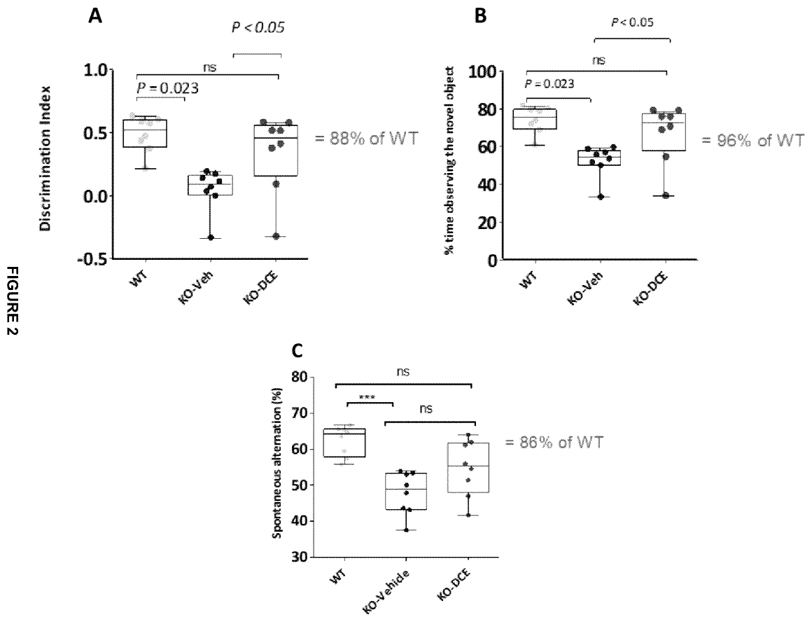

Cognitive performance results indicated that DCE-treated CrT KO mice spent more time observing the novel object than the vehicle-treated CrT KO mice (one-way ANOVA, p<0.01, post-hoc Tukey test p<0.05; FIGS. 2A & 2B) and showed no difference in observation time compared with WT mice (p=0.406). Vehicle-CrT KO mice spent less time compared with WT mice (p<0.01) (FIGS. 2A & 2B). Strikingly, the median time obtained for the DCE-treated group was 96% of the WT median value. In the Y-maze, which is the single variable with the highest accuracy in discriminating WT and KO mice (Raffaele M, et al. Novel translational phenotypes and biomarkers for creatine transporter deficiency. Brain Commun, (2020)), there was a significant reduction of alternation rate shown by vehicle-CrT KO mice (48%) compared to the WT (62%; one-way ANOVA, p<0.001, post-hoc Tukey test p<0.001). The median alternation rate was higher in DCE-treated CrT KO mice and did not differ from WT mice (86%, P=0.058; FIG. 2C). Memory abilities were assessed using the MWM. No positive effects of DCE administration were detected, suggesting that DCE treatment ameliorates some of cognitive deficits seen in CrT KO mice.

Right after behavioral testing, animals were sacrificed. Label-free shotgun proteomics was carried out for each mouse in the CTD, WT and Veh groups on cortex, hippocampus, cerebellum, and brainstem with muscle tissue as control. High-resolution tandem mass spectrometry on the 120 biological samples generated BIG dataset of 7,006,153 MS/MS spectrand the monitoring of the abundance of 4,035 proteins. Following unsupervised filtering and normalization (Hachim M Y, Hachim I Y, Talaat I M, Yakout N M, Hamoudi R. M1 Polarization Markers Are Upregulated in Basal-Like Breast Cancer Molecular Subtype and Associated With Favorable Patient Outcome. Front Immunol 11, 560074 (2020)), proteins significantly affected by CrT deficiency and DCE treatment were identified using reproducibility-optimized statistical testing (Suomi T, Seyednasrollah F, Jaakkola M K, Faux T, Elo L L. ROTS: An R package for reproducibility-optimized statistical testing. PLoS Comput Biol 13, e1005562 (2017)). A total of 376, 322, 163, and 323 proteins across the cortex, cerebellum, brainstem and hippocampus, respectively, were differentially abundant between WT mice compared with vehicle CrT KO mice. The abundances of 320, 416, 279, and 321 proteins were significantly altered by DCE treatment compared with vehicle CrT KO mice in the same tissues, while 413, 223, 176 and 175 proteins were differentially detected in DCE CrT KO mice compared with WT mice. Protein overlaps were observed in the DCE vs vehicle, and WT vs vehicle groups with the highest degree in the cerebellum (153 proteins), hippocampus (126 proteins) and cortex (98 proteins) and lower levels in brainstem (53 proteins). The proteins significantly altered by CrT deficiency compared to WT, and by the treatment compared to vehicle, were then selected for pathway analysis using gene set enrichment analysis. A multivariate statistical model comparing WT and vehicle CrT KO mice vehicle, and DCE-treated CrT KO mice and vehicle CrT KO mice identified key proteins (FIG. 1).

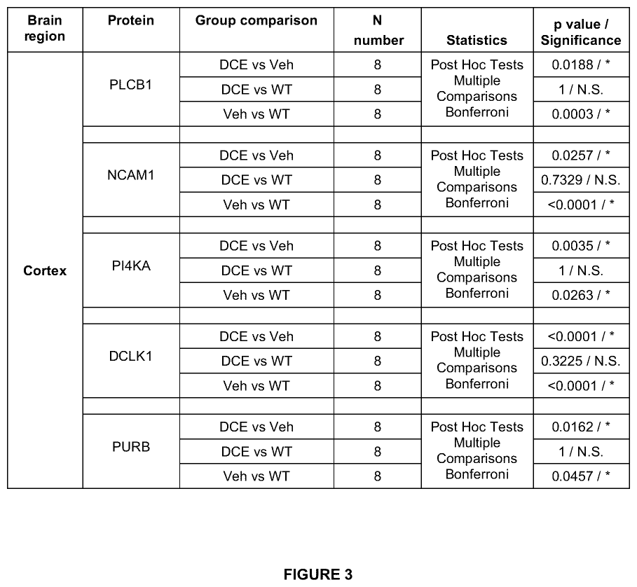

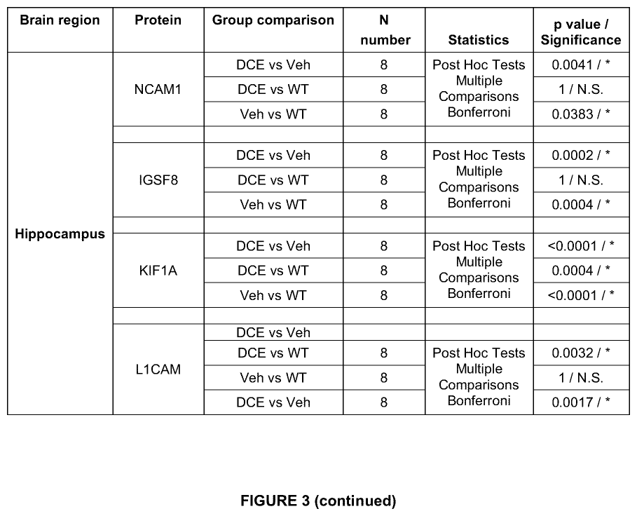

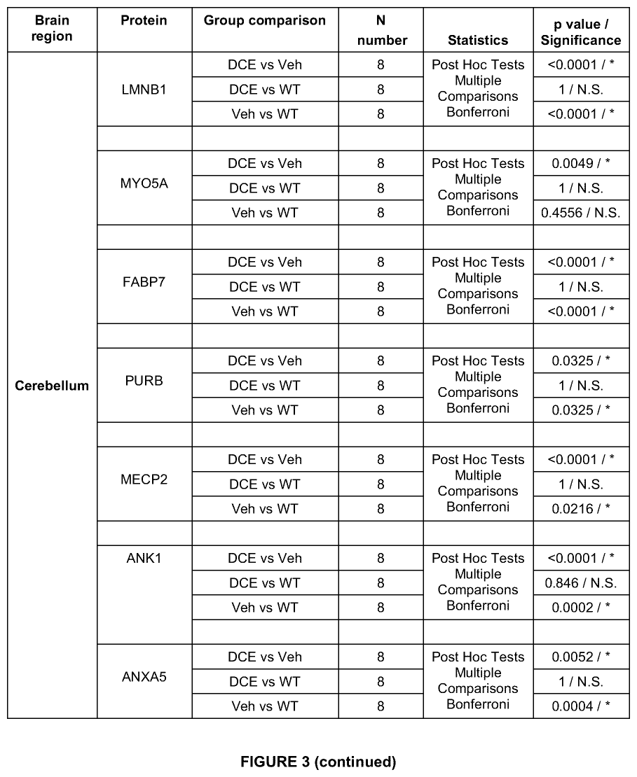

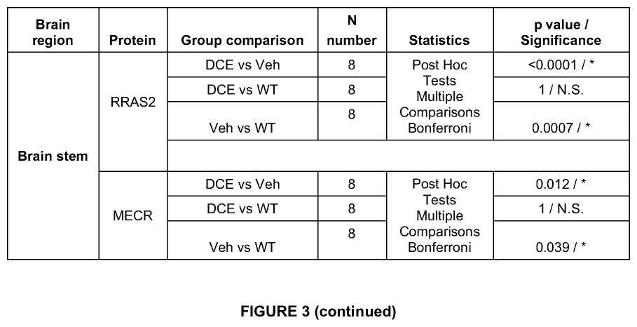

Amongst the proteins affected by either the CrT deficiency or DCE treatment, 14 key are related to both conditions (WT vs Veh, DCE vs Veh) across the 4 different brain regions (FIG. 3). Noteworthily, 13 proteins are related to autism spectrum disorders (MECP2, KIF1A, PLCB1, NCAM1, ANXA5), bipolar disorder (FABP7, NCAM1), axonal neuropathy, affective disorder (DCLK1), intellectual disability (KIF1A, L1CAM), regulation of neurite outgrowth (IGSF8), leukodystrophy (LMNB1), cerebellar ataxia (LMNB1), abnormal behavior (PI4KA), epileptic encephalopathies (PLCB1, MYO5A), and neurodegenerative diseases (ANK1). Whilst the abundance of PLCB1 in the cortex (p=0.0003), MeCP2 (p=0.022), ANK1 (p=0.0001), ANXA5 (P=0.00004) in the cerebellum and IGSF8 (P=0.00036) in the hippocampus were significantly downregulated in vehicle Crt KO mice compared with WT mice, DCE treatment significantly rescued their levels in the same brain regions (FIG. 3). In contrast, NCAM1 (P=1.38E-07), PI4KA (P=0.026), DCLK1 (P=3E-15) and PURB (P=0.045) in the cortex, KIF1A (P=2E-24), NCAM1 (P=0.038), L1CAM (P=0.001) in the hippocampus, and LMNB1 (P=1E-05), FABP7 (P=8.30E-05), and PURB (P=0.03) in the cerebellum were significantly upregulated in Crt KO mice compared with WT mice, but were unchanged between WT and DCE-treated CrT KO mice (FIG. 3).

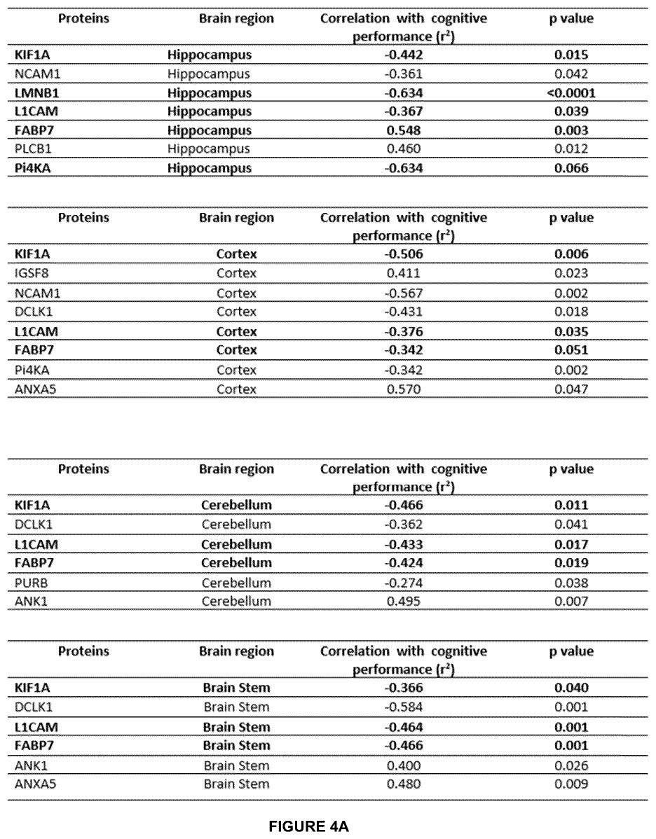

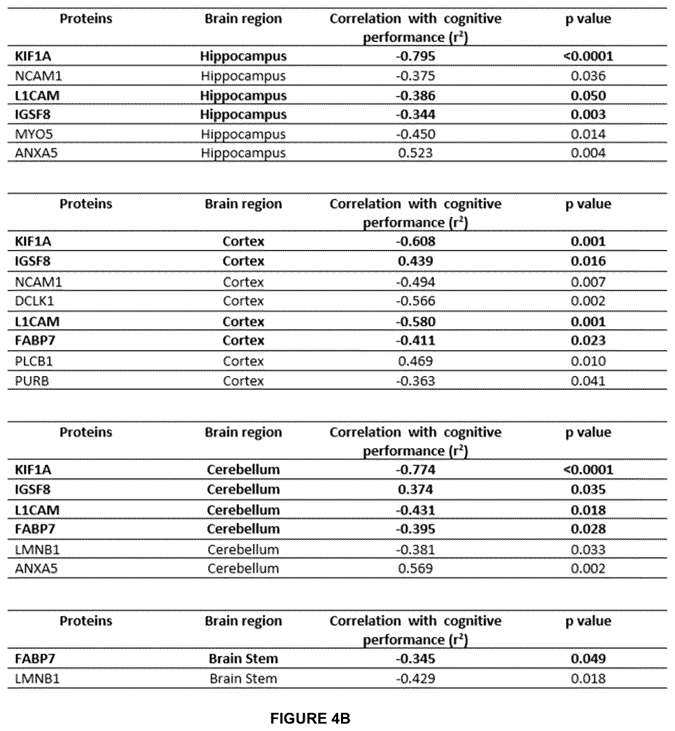

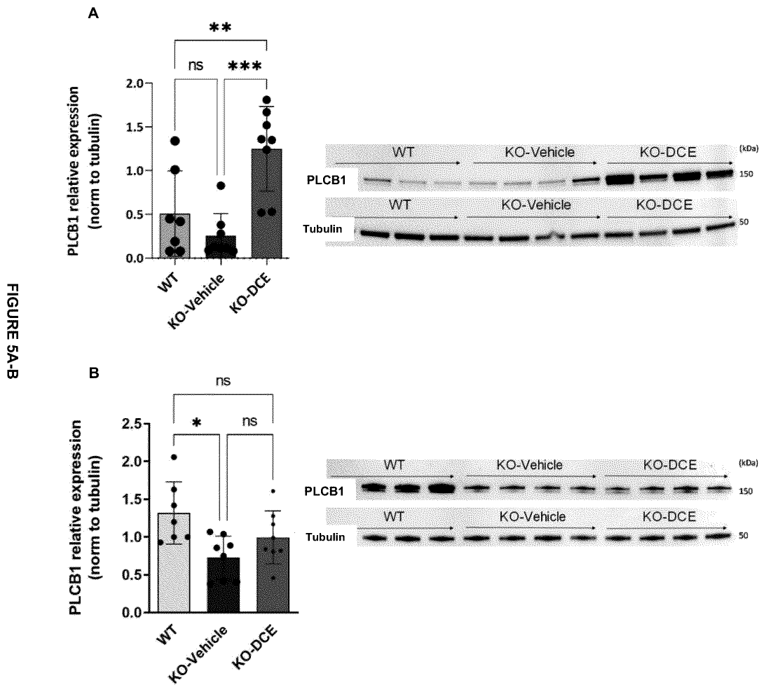

A stepwise regression model testing the effect of each of these proteins with the cognitive outcome identified proteins that may influence cognitive function. Several proteins were significantly correlated with the cognitive performance in discrimination index (n=11, FIG. 4A) and in Y-maze (n=11, FIG. 4B) tests. KIF1A, kinesin that transports synaptic vesicle precursors, showed the strongest correlation with the Y-maze alternation test (hippocampus (r²=−0.795, p<0.0001), cortex (r²=−0.608, p=0.001), cerebellum (r²=0.774, p<0.0001)). The magnitude of this correlation decreases in the discrimination index to −0.442 (p=0.015), −0.566 (p=0.006), −0.466 (p=0.011) and −0.366 (p=0.040) in the hippocampus, cortex, cerebellum and brain stem, respectively. A strong correlation for LMNB1, involved in cerebellar ataxia and adult autosomal dominant leukodystrophy (r²=−0.634 (p<0.0001)) as well as for PI4KA (r²=−0.634, p=0.066) was observed with the cognitive performance in the discrimination index test in the hippocampus. An

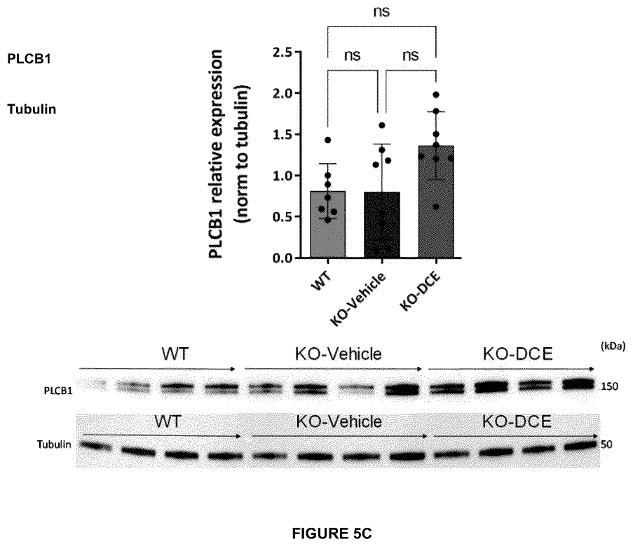

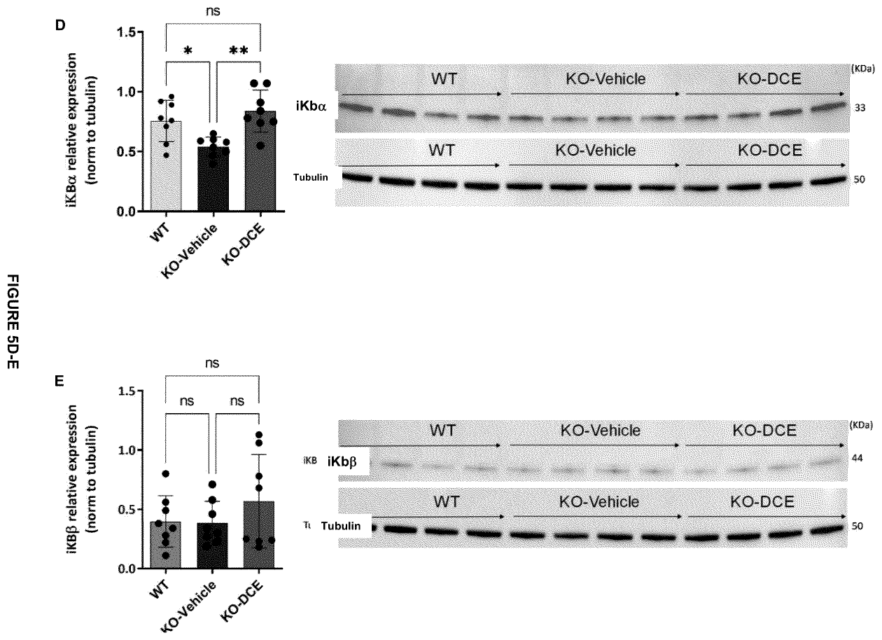

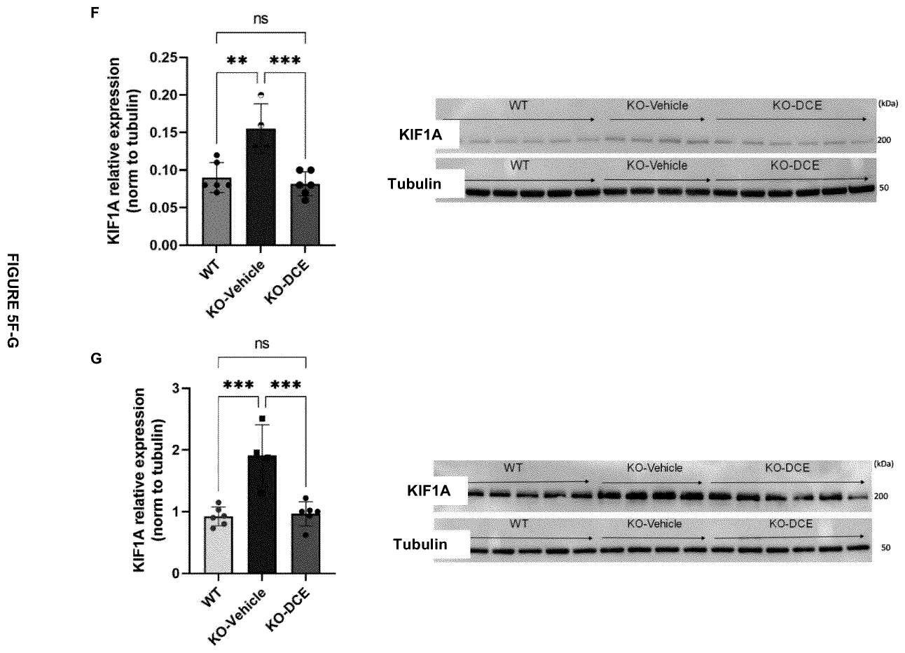

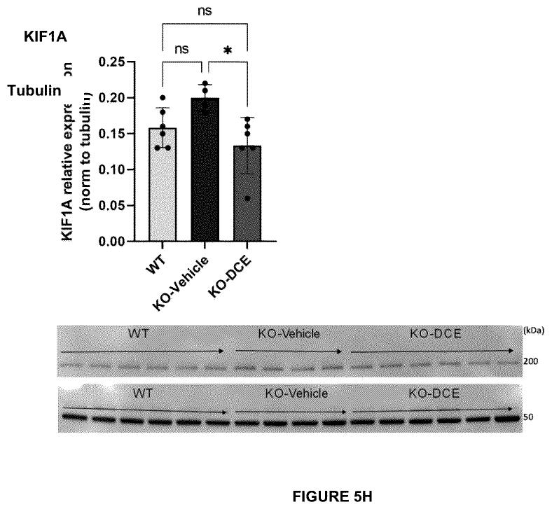

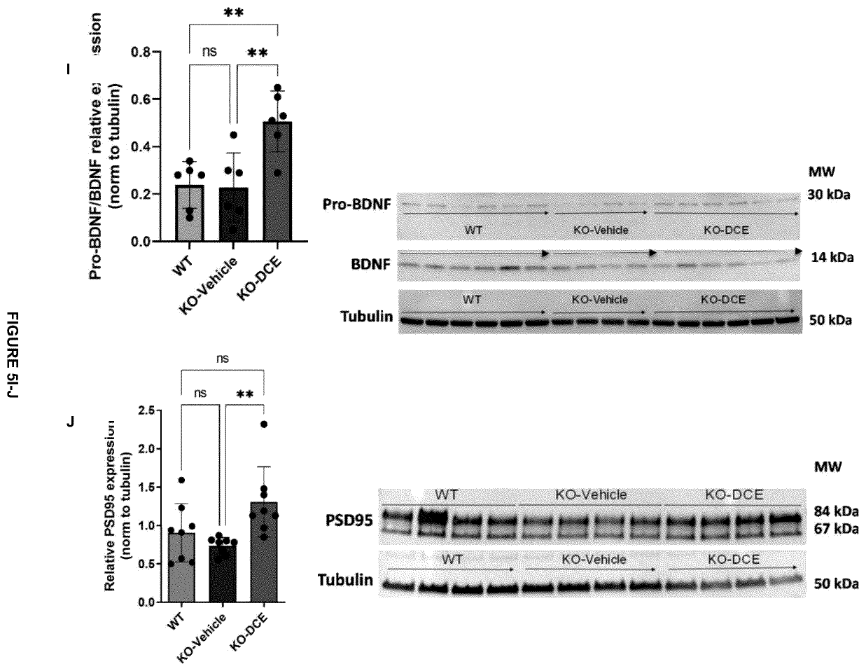

KIF1A mutations were found in patients showing a severe neurodevelopmental disorder with some Rett-like features characterized by MeCP2 dysregulation (Wang J, Zhang Q, Chen Y, Yu S, Wu X, Bao X. Rett and Rett-like syndrome: Expanding the genetic spectrum to KIF1And GRIN1 gene. Mol Genet Genomic Med. 2019 November; 7(11):e968. doi: 10.1002/mgg3.968. Epub 2019 Sep. 11. PMID: 31512412; PMCID: PMC6825848.). A possible interaction between brain derived neurotrophic factor (BDNF) and KIF1A was suggested. Furthermore, BDNF associated to MeCP2 and PLCB1 plays a role in learning memory in animal models of neurodegenerative diseases (Kondo M, Takei Y, Hirokawa N. Motor protein KIF1A is essential for hippocampal synaptogenesis and learning enhancement in an enriched environment. Neuron 73, 743-757 (2012)). Due to their major role in driving cognitive function the possible interplay between KIF1A, PLCB1 and MeCP2 was investigated. Their immunoprecipitation from cortex and hippocampus extracts with anti-KIF1Antibodies was revealed by mass spectrometry and confirmed by Western-blot. Recombinant PLCB1 and MeCP2 isoform1 interact in sandwich ELISA. The abundance of PLCB1 was confirmed by Western blot (Yang Y R, et al. Primary phospholipase C and brain disorders. Adv Biol Regul 61, 80-85 (2016); Lo Vasco V R, Longo L, Polonia P. Phosphoinositide-specific Phospholipase C β1 gene deletion in bipolar disorder affected patient. J Cell Commun Signal 7, 25-29 (2013) et al. (FIG. 5A) which decreased in the hippocampus and cortex of vehicle CrT KO mice, while DCE treated CrT KO mice had significant increase of PLCB1 in cortex (p=0.0004) compared to hippocampus and cerebellum tissues (FIGS. 5A, 5B & 5C). This upregulation of PLCB1 by DCE in brain cortex might lead to production of inositol-1,4,5-triphosphate (IP3) and diacylglycerol (Rusciano I, et al. Location-dependent role of phospholipase C signaling in the brain: Physiology and pathology. Adv Biol Regul 79, 100771 (2021)). In turn, IP3 activates PKCα and modulates NF-κB pathway via the dysregulation of one of its inhibitor, the IκBα protein, thereby modifying the NF-κB inducible genes. IκBα abundance was evaluated to determine if DCE-treatment related increase in PLCB1 affects downstream targets of this pathway. Western blotting indicated significant downregulation of IκBα (p<0.05), but not of IκBβ in brain cortex of vehicle CrT KO mice, while DCE treatment reconstituted the expression of the IκBα protein level (p=0.008) (FIGS. 5D & 5E), suggesting that PLCB1 expression mediates regulation of the NF-κB pathway in CrT KO mice.

KIF1A been found to participate in vesicular transport. Vesicles containing neurotrophin BDNF were implicated to be under KIF1A control (Kondo M, Takei Y, Hirokawa N. Motor protein KIF1A is essential for hippocampal synaptogenesis and learning enhancement in an enriched environment. Neuron. 2012; 73:743-757). BDNF is produced in neocortex throughout brain development, accelerating overall redistribution of cortical neurons. In CTD, DCE-rescued KIF1A in vehicle CrT KO mice (FIG. 5F-H) resulted in higher Pro-BDNF/BDNF level (p=0.0043, FIG. 5I) which is linked to cognitive function improvement.

KIF1A overexpression in the developing brain of CrT KO mice likely leads to non-functional synaptic proteins contributing to cognitive and memory impairments. It was found that in the brain cortex the presynaptic IgSF8 protein abundance, reported as a critical regulator of the brain microcircuit and neuronal function (Apóstolo N, Smukowski S N, Vanderlinden J, et al. Synapse type-specific proteomic dissection identifies IgSF8 as a hippocampal CA3 microcircuit organizer. Nat Commun. 2020; 11(1):5171. Published 2020 Oct. 14. doi:10.1038/s41467-020-18956-x) and the postsynaptic density protein PS95 were altered in vehicle CrT KO mice compared with WT mice, but significantly upregulated by the DCE treatment (p=0.0096, FIG. 5J). These results point to a KIF1A as a potential key player of the pathogenesis of CTD.

Based on the results presented here, a possible cross-talk of the different proteins mediating cognitive function in CTD can be proposed with KIF1A, PLCB1, MeCP2 emerging as key molecules.

This is the first study in-depth describing the molecular mechanisms of CTD cognitive disorders and possible DCE therapy. This work strengthens the therapeutic perspectives of DCE in targeting the cognitive symptoms of CTD, restoration of key molecular players being obtained in several brain subregions.

### REFERENCES

Apóstolo N, Smukowski S N, Vanderlinden J, et al. Synapse type-specific proteomic dissection identifies IgSF8 as a hippocampal CA3 microcircuit organizer. Nat Commun. 2020; 11(1):5171. Published 2020 Oct. 14. doi:10.1038/s41467-020-18956-x
Baroncelli L, et al. A mouse model for creatine transporter deficiency reveals early onset cognitive impairment and neuropathology associated with brain aging. Human Molecular Genetics 25, 4186-4200 (2016)
Baroncelli L, et al. A novel mouse model of creatine transporter deficiency. F1000Res 3, 228 (2014).
Braissant O, Henry H, Beard E, Uldry J. Creatine deficiency syndromes and the importance of creatine synthesis in the brain. Amino Acids 40, 1315-1324 (2011).
Bruun, T. U. J. et al. Treatment outcome of creatine transporter deficiency: international retrospective cohort study. Metabolic Brain Disease 33, 875-884, doi:10.1007/s11011-018-0197-3 (2018).
et al. anJaggumantri, S. et al. Treatment of Creatine Transporter (SLC6A8) Deficiency With Oral S-Adenosyl Methionine as Adjunct to L-arginine, Glycine, and Creatine Supplements. Pediatr Neurol 53, 360-363.e362, doi:10.1016/j.pediatrneurol.2015.05.006 (2015).
et al. DesRoches, C. L. et al. Estimated carrier frequency of creatine transporter deficiency in females in the general population using functional characterization of novel variants missense in the SLC6A8 gene. Gene 565, 187-191, doi:10.1016/j.gene.2015.04.011 (2015).
et al. Hamoudi R A, et al. Differential expression of NF-kappaB target genes in MALT lymphoma with and without chromosome translocation: insights into molecular mechanism. Leukemia 24, 1487-1497 (2010).
et al. Raffaele, M. et al. Novel translational phenotypes and biomarkers for creatine transporter deficiency. Brain Commun., doi:10.1093/braincomms/fcaa089 (2020).
et al. Trotier-Faurion, A. et al. Dodecyl creatine ester and lipid nanocapsule: a double strategy for the treatment of creatine transporter deficiency. Nanomedicine 10, 185-191, doi:10.2217/nnm.13.205 (2015).
Kondo M, Takei Y, Hirokawa N. Motor protein KIF1A is essential for hippocampal synaptogenesis and learning enhancement in an enriched environment. Neuron. 2012; 73(4):743-757. doi:10.1016/j.neuron.2011.12.020
Lancaster M A, Knoblich J A. Generation of cerebral organoids from human pluripotent stem cells. Nat Protoc. 2014; 9(10):2329-2340. doi:10.1038/nprot.2014.158
Lo Vasco, V. R., Longo, L. & Polonia, P. Phosphoinositide-specific Phospholipase C β1 gene deletion in bipolar disorder affected patient. J Cell Commun Signal 7, 25-29, doi:10.1007/s12079-012-0182-2 (2013).
Nassor F, Jarray R, Biard D S F, et al. Long Term Gene Expression in Human Induced Pluripotent Stem Cells and Cerebral Organoids to Model a Neurodegenerative Disease. Front Cell Neurosci. 2020; 14:14. Published 2020 Feb. 11. doi:10.3389/fncel.2020.00014
Pavoni S, Jarray R, Nassor F, et al. Small-molecule induction of Aβ-42 peptide production in human cerebral organoids to model Alzheimer's disease associated phenotypes. PLoS One. 2018; 13(12):e0209150. Published 2018 Dec. 17. doi:10.1371/journal.pone.0209150
Rusciano, I. et al. Location-dependent role of phospholipase C signaling in the brain: Physiology and pathology. Adv Biol Regul 79, 100771, doi:10.1016/j.jbior.2020.100771 (2021).
Salomons, G. S. et al. X-linked creatine-transporter gene (SLC6A8) defect: A new creatine-deficiency syndrome. American Journal of Human Genetics 68, 1497-1500, doi:10.1086/320595 (2001).
Stockler, S., Schutz, P. W. & Salomons, G. S. Cerebral creatine deficiency syndromes: clinical aspects, treatment and pathophysiology. Subcell Biochem 46, 149-166, doi:10.1007/978-1-4020-6486-9_8 (2007).
Suomi T, Seyednasrollah F, Jaakkola M K, Faux T, Elo L L. ROTS: An R package for reproducibility-optimized statistical testing. PLoS Comput Biol 13, e1005562 (2017).
Trotier-Faurion, A. et al. Synthesis and Biological Evaluation of New Creatine Fatty Esters Revealed Dodecyl Creatine Ester as a Promising Drug Candidate for the Treatment of the Creatine Transporter Deficiency. Journal of Medicinal Chemistry 56, 5173-5181, doi:10.1021/jm400545n (2013).
Ullio-Gamboa, G. et al. Dodecyl creatine ester-loaded nanoemulsion as a promising therapy for creatine transporter deficiency. Nanomedicine 14, 1579-1593, doi:10.2217/nnm-2019-0059 (2019).
Valayannopoulos, V. et al. Functional and electrophysiological characterization of four non-truncating mutations responsible for creatine transporter (SLC6A8) deficiency syndrome. Journal of Inherited Metabolic Disease 36, 103-112, doi:10.1007/s10545-012-9495-9 (2013).
van de Kamp, J. M., Mancini, G. M. & Salomons, G. S. X-linked creatine transporter deficiency: clinical aspects and pathophysiology. Journal of Inherited Metabolic Disease 37, 715-733, doi:10.1007/s10545-014-9713-8 (2014).
Wang J, Zhang Q, Chen Y, Yu S, Wu X, Bao X. Rett and Rett-like syndrome: Expanding the genetic spectrum to KIF1And GRIN1 gene. Mol Genet Genomic Med. 2019 November; 7(11):e968. doi:10.1002/mgg3.968. Epub 2019 Sep. 11. PMID: 31512412; PMCID: PMC6825848.
Yang, Y. R. et al. Primary phospholipase C and brain disorders. Adv Biol Regul 61, 80-85, doi:10.1016/j.jbior.2015.11.003 (2016).
Yann, E. et al. Huntingtin phosphorylation governs BDNF homeostasis and improves the phenotype of Mecp2 knockout mice. EMBO Mol Med, doi:10.15252/emmm.201910889 (2020).

1. A method for diagnosing a cerebral creatine deficiency syndrome in an individual in need thereof, said method comprising at least the steps of:
a) measuring an amount of at least one protein selected from BDNF, KIF1A, MeCP2, PLCB1, and a combination thereof, in an isolated biological sample obtained from said individual,
b) comparing the amount measured at step a) with a predetermined reference value,
wherein a difference between the measured amount and the predetermined reference value is indicative of a cerebral creatine deficiency syndrome in said individual.

2. The method according to claim 1, wherein step a) comprises measuring an amount of each protein KIF1A, MeCP2 and PLCB1.

3. The method according to claim 2, wherein step a) comprises further measuring an amount of the protein BDNF.

4. A method for monitoring a therapeutic efficacy of a therapeutic treatment proposed for preventing and/or treating a cerebral creatine deficiency syndrome in an individual in need thereof, said method comprising the steps of:
a) measuring an amount of at least one protein selected from BDNF, KIF1A, MeCP2, PLCB1, and a combination thereof, in an isolated biological sample obtained from said individual before administration of said therapeutic treatment,
b) measuring an amount of at least one protein selected from BDNF, KIF1A, MeCP2, PLCB1, and a combination thereof, in an isolated biological sample obtained from said individual after administration of said therapeutic treatment, the protein or combination of proteins of step a) and b) being the same,
c) comparing the amounts measured at step a) with the amounts measured at step b),
wherein a difference between the measured amounts at step a) and at step b) is indicative of a therapeutic efficacy of said therapeutic treatment on said cerebral creatine deficiency syndrome.

5. The method according to claim 4, wherein steps a) and b) comprise measuring an amount of each protein KIF1A, MeCP2 and PLCB1.

6. The method according to claim 5, wherein steps a) and b) comprise further measuring an amount of the protein BDNF.

7. A method for selecting a candidate therapeutic agent for preventing and/or treating a cerebral creatine deficiency syndrome in an individual in need thereof, said method comprising the steps of:
a) measuring an amount of at least one protein selected from BDNF, KIF1A, MeCP2, PLCB1, and a combination thereof, in an isolated biological sample obtained from a biological model of a cerebral creatine deficiency syndrome before contacting said biological model with said candidate therapeutic agent,
b) measuring an amount of at least one protein selected from BDNF, KIF1A, MeCP2, PLCB1, and a combination thereof, in an isolated biological sample obtained from said biological model of step a) after contacting said biological model with said candidate therapeutic agent, the protein or combination of proteins of step a) and b) being the same,
c) comparing the amounts measured at step a) with the amounts measured at step b), and
d) selecting a candidate therapeutic agent for which a difference between the measured amounts obtained at step a) and at step b) is indicative of a therapeutic efficacy of said candidate therapeutic agent on said cerebral creatine deficiency syndrome.

8. The method according to claim 7, wherein steps a) and b) comprise measuring an amount of each protein KIF1A, MeCP2 and PLCB1.

9. The method according to claim 8, wherein steps a) and b) comprise further measuring an amount of the protein BDNF.

10. A method for monitoring an evolution of a cerebral creatine deficiency syndrome in an individual in need thereof, said method comprising the steps of:
a) measuring an amount of at least one protein selected from BDNF, KIF1A, MeCP2, PLCB1, and a combination thereof, in an isolated biological sample obtained from said individual at a first time,
b) measuring an amount of at least one protein selected from BDNF, KIF1A, MeCP2, PLCB1, and a combination thereof, in an isolated biological sample obtained from said individual at a second time, subsequent to the first time, the protein or combination of proteins of step a) and b) being the same,
c) comparing the measured amounts obtained at step a) and at step b),
wherein a difference between the measured amounts may be indicative of an improvement or an aggravation of the cerebral creatine deficiency syndrome in said individual.

11. The method according to claim 10, wherein steps a) and b) comprise measuring an amount of each protein KIF1A, MeCP2 and PLCB1.

12. The method according to claim 11, wherein steps a) and b) comprise further measuring an amount of the protein BDNF.

13. The method according to claim 1, further comprises a measure of an amount of at least one protein from the group FABP7, LMNB1, and IGSF8, and combinations thereof.

14. The method according to claim 1, further comprises a measure of an amount of at least one protein from the group NCAM1, ANXA5, DCLK1, L1CAM, PI4K-A, MYO5, ANK1 and PURB, and combinations thereof.

15. The method according to claim 1, wherein the cerebral creatine deficiency syndrome is a creatine transporter (CRTR) deficiency.

16. The method according to claim 1, wherein the biological sample is selected from the group consisting of blood, plasma, serum, cerebrospinal fluid, or is a brain organoid prepared by dedifferentiation and reprogramming of fibroblast cells obtained from said individual.

17. Use of at least one protein selected from BDNF, KIF1A, MeCP2, PLCB1, and a combination thereof, as biomarker of a cerebral creatine deficiency syndrome.

18. The use according to claim 17, wherein the biomarker is a set of proteins comprising KIF1A, MeCP2, and PLCB1.

19. The use according to claim 18, wherein the biomarker further comprises the protein BDNF.

20. A biomarker for use in a method for diagnosing a cerebral creatine deficiency syndrome, wherein the biomarker comprises at least one protein selected from BDNF, KIF1A, MeCP2, PLCB1, and a combination thereof.

21. The biomarker for use according to claim 20, wherein the biomarker is a set of proteins comprising KIF1A, MeCP2, and PLCB1.

22. The biomarker for use according to claim 21, wherein the biomarker further comprises the protein BDNF.

23. The use according to claim 17 or the biomarker for use according to claim 20, further comprising at least one protein from the group FABP7, LMNB1, and IGSF8, and combinations thereof, and/or of at least one protein from the group NCAM1, ANXA5, DCLK1, L1CAM, PI4K-A, MYO5, ANK1 and PURB, and combinations thereof.

24. The use according to claim 17 or the biomarker for use according to claim 20, wherein the cerebral creatine deficiency syndrome is a creatine transporter (CRTR) deficiency.

25. A kit for diagnosing a cerebral creatine deficiency syndrome, said kit comprising means for measuring an amount of at least one protein selected from BDNF, KIF1A, MeCP2, PLCB1, and a combination thereof, in an isolated biological sample.

26. The kit according to claim 25, wherein the kit comprises means for measuring an amount of each protein KIF1A, MeCP2, and PLCB1.

27. The kit according to claim 26, wherein the kit comprises means for measuring an amount of the protein BDNF.

28. The kit according to claim 25, wherein the means for determining the amount of said protein are configured for performing an immunoassay and/or a mass-spectrometric-based assay.

29. The kit according to claim 25, comprising an instruction to compare the measured amounts of the proteins with predetermined reference values.
